SEQUENCE LISTING (32 sequences)

SEQ ID NO: 1 - protein, 267 aa, Homo sapiens
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 2 - protein, 13 aa, Homo sapiens
SYSMEHFRWGKPV

SEQ ID NO: 3 - protein, 18 aa, Homo sapiens
DEGPYRMEHFRWGSPPKD

SEQ ID NO: 4 - protein, 11 aa, Homo sapiens
YVMGHFRWDRF

SEQ ID NO: 5 - protein, 39 aa, Homo sapiens
SYSMEHFRWGKPVGKKRRPVKVYPNGAEDESAEAFPIEF

SEQ ID NO: 6 - protein, 13 aa, Human immunodeficiency virus
KYKYLESRWNDRM

SEQ ID NO: 7 - DNA, 1295 bp, Homo sapiens
ccttcccctggcccggggagctgctccttgtgctgccgggaaggtcaaagtcccgcgcccaccaggagagctcggcaagtatataaggacagaggagcgcgggaccaagcggcggcgaaggaggggaagaagagccgcgaccgagagaggccgccgagcgtccccgccctcagagagcagcctcccgagacaggcacttgctggattctccaaaagtatctgcagtggctgttccaccaggagagcctcagcctgcctggaagatgccgagatcgtgctgcagccgctcgggggccctgttgctggccttgctgcttcaggcctccatggaagtgcgtggctggtgcctggagagcagccagtgtcaggacctcaccacggaaagcaacctgctggagtgcatccgggcctgcaagcccgacctctcggccgagactcccatgttcccgggaaatggcgacgagcagcctctgaccgagaacccccggaagtacgtcatgggccacttccgctgggaccgattcggccgccgcaacagcagcagcagcggcagcagcggcgcagggcagaagcgcgaggacgtctcagcgggcgaagactgcggcccgctgcctgagggcggccccgagccccgcagcgatggtgccaagccgggcccgcgcgagggcaagcgctcctactccatggagcacttccgctggggcaagccggtgggcaagaagcggcgcccagtgaaggtgtaccctaacggcgccgaggacgagtcggccgaggccttccccctggagttcaagagggagctgactggccagcgactccgggagggagatggccccgacggccctgccgatgacggcgcaggggcccaggccgacctggagcacagcctgctggtggcggccgagaagaaggacgagggcccctacaggatggagcacttccgctggggcagcccgcccaaggacaagcgctacggcggtttcatgacctccgagaagagccagacgcccctggtgacgctgttcaaaaacgccatcatcaagaacgcctacaagaagggcgagtgagggcacagcggggccccagggctaccctcccccaggaggtcgaccccaaagccccttgctctcccctgccctgctgccgcctcccagcctggggggtcgtggcagataatcagcctcttaaagctgcctgtagttaggaaataaaacctttcaaatttcacatccacctctgactttgaatgtaaactgtgtgaataaagtaaaaatacgtagccgtcaaataacagc

SEQ ID NO: 8 - DNA, 1245 bp, Homo sapiens
ccttcccctggcccggggagctgctccttgtgctgccgggaaggtcaaagtcccgcgcccaccaggagagctcggcaagtatataaggacagaggagcgcgggaccaagcggcggcgaaggaggggaagaagagccgcgaccgagagaggccgccgagcgtccccgccctcagagagcagcctcccgagacagagcctcagcctgcctggaagatgccgagatcgtgctgcagccgctcgggggccctgttgctggccttgctgcttcaggcctccatggaagtgcgtggctggtgcctggagagcagccagtgtcaggacctcaccacggaaagcaacctgctggagtgcatccgggcctgcaagcccgacctctcggccgagactcccatgttcccgggaaatggcgacgagcagcctctgaccgagaacccccggaagtacgtcatgggccacttccgctgggaccgattcggccgccgcaacagcagcagcagcggcagcagcggcgcagggcagaagcgcgaggacgtctcagcgggcgaagactgcggcccgctgcctgagggcggccccgagccccgcagcgatggtgccaagccgggcccgcgcgagggcaagcgctcctactccatggagcacttccgctggggcaagccggtgggcaagaagcggcgcccagtgaaggtgtaccctaacggcgccgaggacgagtcggccgaggccttccccctggagttcaagagggagctgactggccagcgactccgggagggagatggccccgacggccctgccgatgacggcgcaggggcccaggccgacctggagcacagcctgctggtggcggccgagaagaaggacgagggcccctacaggatggagcacttccgctggggcagcccgcccaaggacaagcgctacggcggtttcatgacctccgagaagagccagacgcccctggtgacgctgttcaaaaacgccatcatcaagaacgcctacaagaagggcgagtgagggcacagcggggccccagggctaccctcccccaggaggtcgaccccaaagccccttgctctcccctgccctgctgccgcctcccagcctggggggtcgtggcagataatcagcctcttaaagctgcctgtagttaggaaataaaacctttcaaatttcacatccacctctgactttgaatgtaaactgtgtgaataaagtaaaaatacgtagccgtcaaataacagc

SEQ ID NO: 9 - protein, 855 aa, Human immunodeficiency virus
MKVKGTRRNYQHLWRWGTLLLGMLMICSATEKLWVTVYYGVPVWKEATTTLFCASDARAYDTEVHNVWATHACVPTDPNPQEVVLGNVTENFNMWKNNMVEQMQEDIISLWDQSLKPCVKLTPLCVTLNCTDLGKATNTNSSNWKEEIKGEIKNCSFNITTSIRDKIQKENALFRNLDVVPIDNASTTTNYTNYRLIHCNRSVITQACPKVSFEPIPIHYCTPAGFAILKCNNKTFNGKGPCTNVSTVQCTHGIRPIVSTQLLLNGSLAEEEVVIRSDNFTNNAKTIIVQLNESVAINCTRPNNNTRKSIYIGPGRAFHTTGRIIGDIRKAHCNISRAQWNNTLEQIVKKLREQFGNNKTIVFNQSSGGDPEIVMHSFNCRGEFFYCNTTQLFNNTWRLNHTEGTKGNDTIILPCRIKQIINMWQEVGKAMYAPPIGGQISCSSNITGLLLTRDGGTNVTNDTEVFRPGGGDMRDNWRSELYKYKVIKIEPLGIAPTKAKRRVVQREKRAVGIVGAMFLGFLGAAGSTMGAVSLTLTVQARQLLSGIVQQQNNLLRAIEAQQHLLQLTVWGIKQLQARVLAVERYLRDQQLLGIWGCSGKLICTTAVPWNASWSNKSLEDIWDNMTWMQWEREIDNYTNTIYTLLEESQNQQEKNEQELLELDKWASLWNWFSITNWLWYIKIFIMIVGGLVGLRIVFAVLSIVNRVRQGYSPLSFQTRLPVPRGPDRPDGIEEEGGERDRDRSVRLVDGFLALIWEDLRSLCLFSYRRLRDLLLIAARTVEILGHRGWEALKYWWSLLQYWIQELKNSAVSWLNATAIAVTEGTDRVIEVAQRAYRAILHIHRRIRQGLERLLL

SEQ ID NO: 10 - DNA, 2568 bp, Human immunodeficiency virus
atgaaagtgaaggggaccaggaggaattatcagcacttgtggagatggggcaccttgctccttgggatgttgatgatctgtagtgctacagaaaaattgtgggtcacagtttattatggagtacctgtgtggaaagaagcaactaccactctattttgtgcatcagatgctagagcatatgatacagaggtacataatgtttgggccacacatgcctgtgtacccacagaccccaacccacaagaagtagtattgggaaatgtgacagaaaattttaacatgtggaaaaataacatggtagaacagatgcaggaggatataatcagtttatgggatcaaagcctaaagccatgtgtaaaattaaccccactctgtgttactttaaattgcactgatttggggaaggctactaataccaatagtagtaattggaaagaagaaataaaaggagaaataaaaaactgctctttcaatatcaccacaagcataagagataagattcagaaagaaaatgcactttttcgtaaccttgatgtagtaccaatagataatgctagtactactaccaactataccaactataggttgatacattgtaacagatcagtcattacacaggcctgtccaaaggtatcatttgagccaattcccatacattattgtaccccggctggttttgcgattctaaagtgtaataataaaacgttcaatggaaaaggaccatgtacaaatgtcagcacagtacaatgtacacatggaattaggccaatagtgtcaactcaactgctgttaaatggcagtctagcagaagaagaggtagtaattagatctgacaatttcacgaacaatgctaaaaccataatagtacagctgaatgaatctgtagcaattaactgtacaagacccaacaacaatacaagaaaaagtatctatataggaccagggagagcatttcatacaacaggaagaataataggagatataagaaaagcacattgtaacattagtagagcacaatggaataacactttagaacagatagttaaaaaattaagagaacagtttgggaataataaaacaatagtctttaatcaatcctcaggaggggacccagaaattgtaatgcacagttttaattgtagaggggaatttttctactgtaatacaacacaactgtttaataatacatggaggttaaatcacactgaaggaactaaaggaaatgacacaatcatactcccatgtagaataaaacaaattataaacatgtggcaggaagtaggaaaagcaatgtatgcccctcccattggaggacaaattagttgttcatcaaatattacagggctgctattaacaagagatggtggtacaaatgtaactaatgacaccgaggtcttcagacctggaggaggagatatgagggacaattggagaagtgaattatataaatataaagtaataaaaattgaaccattaggaatagcacccaccaaggcaaagagaagagtggtgcagagagaaaaaagagcagtgggaatagtaggagctatgttccttgggttcttgggagcagcaggaagcactatgggcgcagtgtcattgacgctgacggtacaggccagacaattattgtctggtatagtgcaacagcagaacaatttgctgagggctattgaggcgcaacaacatctgttgcaactcacagtctggggcatcaagcagctccaggcaagagtcctggctgtggaaagatacctaagggatcaacagctcctagggatttggggttgctctggaaaactcatttgcaccactgctgtgccttggaatgctagttggagtaataaatctctggaagacatttgggataacatgacctggatgcagtgggaaagagaaattgacaattacacaaacacaatatacaccttacttgaagaatcgcagaaccaacaagaaaagaatgaacaagaattattagaattggataagtgggcaagtttgtggaattggtttagcataacaaactggctgtggtatataaagatattcataatgatagtaggaggcttggtaggtttaagaatagtttttgctgtgctttctatagtgaatagagttaggcagggatactcaccattgtcatttcagacccgcctcccagtcccgaggggacccgacaggcccgacggaatcgaagaagaaggtggagagagagacagagacagatccgttcgattagtggatggattcttagcacttatctgggaagatctgcggagcctgtgcctcttcagctaccgccgcttgagagacttactcttgattgcagcgaggactgtggaaattctggggcacagggggtgggaagccctcaaatattggtggagtctcctgcagtattggattcaggaactaaagaatagtgctgttagctggctcaacgccacagctatagcagtaactgaggggacagatagggttatagaagtagcacaaagagcttatagagctattctccacatacatagaagaattagacagggcttggaaaggcttttgctataa

SEQ ID NO: 11 - protein, 332 aa, Homo sapiens
MVNSTHRGMHTSLHLWNRSSYRLHSNASESLGKGYSDGGCYEQLFVSPEVFVTLGVISLLENILVIVAIAKNKNLHSPMYFFICSLAVADMLVSVSNGSETIIITLLNSTDTDAQSFTVNIDNVIDSVICSSLLASICSLLSIAVDRYFTIFYALQYHNIMTVKRVGIIISCIWAACTVSGILFIIYSDSSAVIICLITMFFTMLALMASLYVHMFLMARLHIKRIAVLPGTGAIRQGANMKGAITLTILIGVFVVCWAPFFLHLIFYISCPQNPYCVCFMSHFNLYLILIMCNSIIDPLIYALRSQELRKTFKEIICCYPLGGLCDLSSRY

SEQ ID NO: 12 - DNA, 999 bp, Homo sapiens
atggtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattatcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagcgggttgggatcatcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggagcgattaccttgaccatcctgattggcgtctttgttgtctgctgggccccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcagatattaa

SEQ ID NO: 13 - protein, 325 aa, Homo sapiens
MNSSFHLHFLDLNLNATEGNLSGPNVKNKSSPCEDMGIAVEVFLTLGVISLLENILVIGAIVKNKNLHSPMYFFVCSLAVADMLVSMSSAWETITIYLLNNKHLVIADAFVRHIDNVFDSMICISVVASMCSLLAIAVDRYVTIFYALRYHHIMTARRSGAIIAGIWAFCTGCGIVFILYSESTYVILCLISMFFAMLFLLVSLYIHMFLLARTHVKRIAALPGASSARQRTSMQGAVTVTMLLGVFTVCWAPFFLHLTLMLSCPQNLYCSRFMSHFNMYLILIMCNSVMDPLIYAFRSQEMRKTFKEIICCRGFRIACSFPRRD

SEQ ID NO: 14 - DNA, 978 bp, Homo sapiens
atgaattcctcatttcacctgcatttcttggatctcaacctgaatgccacagagggcaacctttcaggacccaatgtcaaaaacaagtcttcaccatgtgaagacatgggcattgctgtggaggtgtttctcactctgggtgtcatcagcctcttggagaacatcttggtcataggggccatagtgaagaacaaaaacctgcactcccccatgtacttcttcgtgtgcagcctggcagtggcggacatgctggtgagcatgtccagtgcctgggagaccatcaccatctacctactcaacaacaagcacctagtgatagcagacgcctttgtgcgccacattgacaatgtgtttgactccatgatctgcatttccgtggtggcatccatgtgcagcttactggccattgcagtggataggtacgtcaccatcttctacgccctgcgctaccaccacatcatgacggcgaggcgctcaggggccatcatcgccggcatctgggctttctgcacgggctgcggcattgtcttcatcctgtactcagaatccacctacgtcatcctgtgcctcatctccatgttcttcgctatgctgttcctcctggtgtctctgtacatacacatgttcctcctggcgcggactcacgtcaagcggatcgcggctctgcccggggccagctctgcgcggcagaggaccagcatgcagggcgcggtcaccgtcaccatgctgctgggcgtgtttaccgtgtgctgggccccgttcttccttcatctcactttaatgctttcttgccctcagaacctctactgctctcgcttcatgtctcacttcaatatgtacctcatactcatcatgtgtaattccgtgatggaccctctcatatatgccttccgcagccaagagatgcggaagacctttaaggagattatttgctgccgtggtttcaggatcgcctgcagctttcccagaagggattaa

SEQ ID NO: 15 - protein, 482 aa, Human immunodeficiency virus
SATEKLWVTVYYGVPVWKEATTTLFCASDARAYDTEVHNVWATHACVPTDPNPQEVVLGNVTENFNMWKNNMVEQMQEDIISLWDQSLKPCVKLTPLCVTLNCTDLGKATNTNSSNWKEEIKGEIKNCSFNITTSIRDKIQKENALFRNLDVVPIDNASTTTNYTNYRLIHCNRSVITQACPKVSFEPIPIHYCTPAGFAILKCNNKTFNGKGPCTNVSTVQCTHGIRPIVSTQLLLNGSLAEEEVVIRSDNFTNNAKTIIVQLNESVAINCTRPNNNTRKSIYIGPGRAFHTTGRIIGDIRKAHCNISRAQWNNTLEQIVKKLREQFGNNKTIVFNQSSGGDPEIVMHSFNCRGEFFYCNTTQLFNNTWRLNHTEGTKGNDTIILPCRIKQIINMWQEVGKAMYAPPIGGQISCSSNITGLLLTRDGGTNVTNDTEVFRPGGGDMRDNWRSELYKYKVIKIEPLGIAPTKAKRRVVQREKR

SEQ ID NO: 16 - protein, 346 aa, Human immunodeficiency virus
AVGIVGAMFLGFLGAAGSTMGAVSLTLTVQARQLLSGIVQQQNNLLRAIEAQQHLLQLTVWGIKQLQARVLAVERYLRDQQLLGIWGCSGKLICTTAVPWNASWSNKSLEDIWDNMTWMQWEREIDNYTNTIYTLLEESQNQQEKNEQELLELDKWASLWNWFSITNWLWYIKIFIMIVGGLVGLRIVFAVLSIVNRVRQGYSPLSFQTRLPVPRGPDRPDGIEEEGGERDRDRSVRLVDGFLALIWEDLRSLCLFSYRRLRDLLLIAARTVEILGHRGWEALKYWWSLLQYWIQELKNSAVSWLNATAIAVTEGTDRVIEVAQRAYRAILHIHRRIRQGLERLLL

SEQ ID NO: 17 - protein, 25 aa, Human immunodeficiency virus
GGGDMRDNWRSELYKYKVVKIEPLG

SEQ ID NO: 18 - protein, 25 aa, Human immunodeficiency virus
FSADVAELYRLELGDYKLVEITPIG

SEQ ID NO: 19 - protein, 25 aa, Human immunodeficiency virus
VSAEVSELYRLEIGDYKLVEITPIG

SEQ ID NO: 20 - protein, 25 aa, Human immunodeficiency virus
FSAEVAELYRLELGDYKLIEVTPIG

SEQ ID NO: 21 - protein, 25 aa, Human immunodeficiency virus
MSAEVAELYRLELGDYKLVEITPIG

SEQ ID NO: 22 - protein, 8 aa, Human immunodeficiency virus
ASTTTNYT

SEQ ID NO: 23 - protein, 13 aa, Human immunodeficiency virus
RNSSSSGSSGAGQ

SEQ ID NO: 24 - protein, 13 aa, Human immunodeficiency virus
MRDNWRSELYKYK

SEQ ID NO: 25 - protein, 12 aa, Human immunodeficiency virus
RDNWRSELYKYK

SEQ ID NO: 26 - protein, 14 aa, Human immunodeficiency virus
KYKYLESRWNDRMD

SEQ ID NO: 27 - protein, 17 aa, Human immunodeficiency virus
KYKYLESRWNDRMDGGG

SEQ ID NO: 28 - protein, 17 aa, Human immunodeficiency virus
GGGDMRDNWRSELYKYK

SEQ ID NO: 29 - protein, 12 aa, Human immunodeficiency virus
KYKYLESRWNDR

SEQ ID NO: 30 - protein, 14 aa, Human immunodeficiency virus
DMRDNWRSELYKYK

SEQ ID NO: 31 - protein, 8 aa, Human immunodeficiency virus
  VARIANT 3, 4, 5, 6, 7: Xaa = Any Amino Acid
WRXXXXXY

SEQ ID NO: 32 - protein, 8 aa, Human immunodeficiency virus
GPGRAFVT

(19) United States
(12) Patent Application Publication
(10) Pub. No.: US 2010/0009340 A1
(43) Pub. Date: Jan. 14, 2010

(54) COMPOSITIONS AND METHODS FOR THE ANALYSIS AND TREATMENT OF AIDS WASTING SYNDROME AND IMMUNE CELL DYSFUNCTION

(75) Inventor: Stephen Dominy, Novato, CA (US)

Correspondence Address: MORRISON & FOERSTER LLP, 425 MARKET STREET, SAN FRANCISCO, CA, 94105-2482, US

(73) Assignee: The Regents of the University of California, Oakland, CA (US)

(21) Appl. No.: 12/294,422

(22) PCT Filed: Apr. 5, 2007
(86) PCT No.: PCT/US07/08574

§ 371 Date: Jun. 19, 2009

Related U.S. Application Data
(60) Provisional application No. 60/789,827, filed on Apr. 5, 2006.

(51) Int. Cl. C12Q 1/70

(52) U.S. Cl. 435/5

(57) ABSTRACT

The present invention relates to aberrant cell signaling by the HIV type I envelope glycoprotein. In particular, the present invention provides compositions and methods for identification of inhibitors of melanocortin receptor pathway stimulation by HIV-I gp120 and its degradation products. The inhibitors so identified are contemplated to be suitable for treating HIV-related cachexia, T cell hyperactivation and central nervous system disease.

The present application claims the benefit of U.S. Provisional Application No. 60/789,827, filed Apr. 5, 2006, herein incorporated by reference.

### FIELD OF THE INVENTION

The present invention relates to aberrant cell signaling by the HIV type I envelope glycoprotein. In particular, the present invention provides compositions and methods for identification of inhibitors of melanocortin receptor pathway stimulation by HIV-1 gp120 and its degradation products. The inhibitors so identified are contemplated to be suitable for treating HIV-related cachexia, T cell hyperactivation, and central nervous system disease.

### BACKGROUND OF THE INVENTION

An involuntary and progressive loss in body weight is a symptom of HIV infection (HIV-related cachexia). As defined by the Centers for Disease Control, when such weight loss amounts to greater than a ten percent (10%) loss in baseline body weight and is combined with either chronic diarrhea or chronic weakness and fever in the absence of a secondary infection, the weight loss is classified as HIV-related wasting syndrome (e.g., AIDS wasting syndrome). The wasting syndrome is known to play a major role in the decreased quality of life of AIDS patients, and contributes to morbidity and mortality in patients infected with HIV. Classic AIDS wasting syndrome was commonplace prior to the introduction of highly active antiretroviral therapy (HAART). Even so, a significant number of patients still suffer from HIV-related cachexia and wasting syndrome (Hoffmann, Rockstroh, Kamps et al., HIV Medicine 2006, website). Moreover, weight loss remains an independent risk factor for mortality, even in the HAART era (Tang et al., J AIDS, 31:230-236, 2002). Patients with classic wasting syndrome also have an elevated risk of opportunistic infection, (Dworkin and Williamson, J AIDS, 33:267-273, 2003) and may experience cognitive impairment (Dolan et al., J AIDS, 34:155-164, 2003).

Various treatment regimens have been investigated, including alimentation (enteral and parenteral), appetite stimulants, anabolic agents, cytokine modulators and fatty acid supplements with limited success. Thus what is needed in the art is an alternative treatment for HIV-related cachexia and the wasting syndrome.

### SUMMARY OF THE INVENTION

The present invention relates to aberrant cell signaling by the HIV type I envelope glycoprotein. In particular, the present invention provides compositions and methods for identification of inhibitors of melanocortin receptor pathway stimulation by HIV-1 gp120 and its degradation products. The inhibitors so identified are contemplated to be suitable for treating HIV-related cachexia, T cell hyperactivation and central nervous system disease.

In particular, the present invention provides methods for detecting antibodies in a sample, comprising: providing i) a sample, wherein the sample comprises antibodies; and ii) a polypeptide comprising an HIV-1 melanotropin epitope; contacting the polypeptide with the sample under conditions suitable for binding the antibodies to the HIV-1 melanotropin epitope of the polypeptide; and detecting HIV-1 melanotropin epitope-reactive antibodies bound to the polypeptide. In some embodiments, the detecting comprises an enzyme-linked immunosorbent assay, and/or a Western blot. In some preferred embodiments, the sample is from an HIV-1 infected patient. In further embodiments, the sample is from an AIDS vaccine recipient (e.g., whom may or may not be HIV-1-positive). In some embodiments, the methods further comprise a step of providing a prognosis of developing AIDS wasting disease to the subject based on a reduced or absent level of the HIV-1 melanotropin epitope-reactive antibodies. In other embodiments, the methods further comprise a step of providing a prognosis of developing HIV-1 T regulatory cell dysfunction to the subject based on a reduced or absent level of the HIV-1 melanotropin epitope-reactive antibodies. In some preferred embodiments, the methods further comprise a step of providing a prognosis of contracting HIV-1 to the AIDS vaccine recipient based on a reduced or absent level of the HIV-1 melanotropin epitope-reactive antibodies. In other embodiments, the sample is from a hybridoma cell culture supernatant. In some embodiments the polypeptide comprises a 50 kDa fragment of HIV-1 gp120 produced by cleavage with thrombin. In other embodiments, the polypeptides comprises a carrier (e.g., BSA, KLH, etc.). In some particularly preferred embodiments, the polypeptide comprises the amino acid sequence set forth in SEQ ID NO:17 or SEQ ID NO:24. In other preferred embodiments, the polypeptide comprises an HIV-1 gp120 protein. Also provided are methods that further comprise a step of detecting antibodies that neutralize HIV-1 in an in vitro assay.

Additionally, the present invention provides kits for detecting antibodies in a sample, comprising: a polypeptide comprising an HIV-1 melanotropin epitope; and instructions for detecting HIV-1 melanotropin epitope-reactive antibodies in a sample. In some embodiments, the kits further comprise a negative control polypeptide. In other embodiments, the kits further comprise a positive control antibody and a negative control antibody. In some preferred embodiments, the polypeptide comprises the amino acid sequence set forth in SEQ ID NO:17 or 24.

Moreover the present invention provides methods for identifying a compound as an inhibitor of HIV-1 melanotropin epitope stimulation of a human melanocortin receptor (MCR), comprising; providing: i) a cell line expressing a human MCR; ii) a polypeptide comprising an HIV-1 melanotropin epitope; and a iii) compound; contacting the cell line with the polypeptide in the presence and absence of the compound under conditions suitable for stimulating the human MCR with the polypeptide, wherein stimulation of the human MCR in the absence but not the presence of the compound indicates that the compound is an inhibitor of the HIV-1 melanotropin epitope. In some embodiments, the methods further comprise identifying the compound as an inhibitor of human neuropeptide hormone stimulation of human MCR by contacting the cell line with a human neuropeptide hormone in the presence and absence of the compound under conditions suitable for stimulating the MCR with the human neuropeptide hormone, wherein stimulation of the human MCR in the absence but not the presence of the compound indicates that the compound is an inhibitor of the human neuropeptide hormone. In some preferred embodiments, the human MCR is MC4R. In other preferred embodiments, the human MCR is selected from the group consisting of MC1R, MC2R, MC3R and MC5R. In some particularly preferred embodiments, the cell line is a transgenic cell line expressing the MC4R. In some preferred embodiments, the polypeptide comprises the amino acid sequence set forth in SEQ ID NO:17 or 24. Also provided by the present invention are embodiments in which the human neuropeptide hormone is selected from the group consisting of alpha-melanocyte stimulating hormone (alpha-MSH), adrenocorticotrophin (ACTH), beta-melanocyte stimulating hormone (beta-MSH) and gamma-melanocyte stimulating hormone (gamma-MSH).

### DESCRIPTION OF THE FIGURES

FIG. 1A shows the amino acid sequence of the pro-opiomelanocortin (POMC) preproprotein (SEQ ID NO:1). FIG. 1B shows an alignment of the POMC-derived melanocortin receptor ligands alpha-melanocyte stimulating hormone (alpha-MSH or alpha-melanotropin set forth as SEQ ID NO:2), beta-melanocyte stimulating hormone (beta-MSH or beta-melanotropin set forth as SEQ ID NO:3), gamma-melanocyte stimulating hormone (gamma-MSH or gamma-melanotropin set forth as SEQ ID NO:4), adrenocorticotropic hormone (ACTH or adrenocorticotropin set forth as SEQ ID NO:5) with a fragment of HIV-1SF2 gp120 shown in the reverse orientation (K13M peptide set forth as SEQ ID NO:6). As shown in this alignment, tyrosine (Y), arginine (R) and tryptophan (W) residues are conserved in all five sequences.

FIG. 2A shows the cDNA sequence of the POMC transcript variant 1 (SEQ ID NO:7) from GENBANK Accession No. NM_{—}001035256. FIG. 2B shows the cDNA sequence of the POMC transcript variant 2 (SEQ ID NO:8) from GENBANK Accession No. NM_{—}000939. The longer variant 1 contains additional sequence in the 5′ untranslated region (UTR) as compared to the shorter variant 2, although both cDNA sequences encode the same POMC protein sequence.

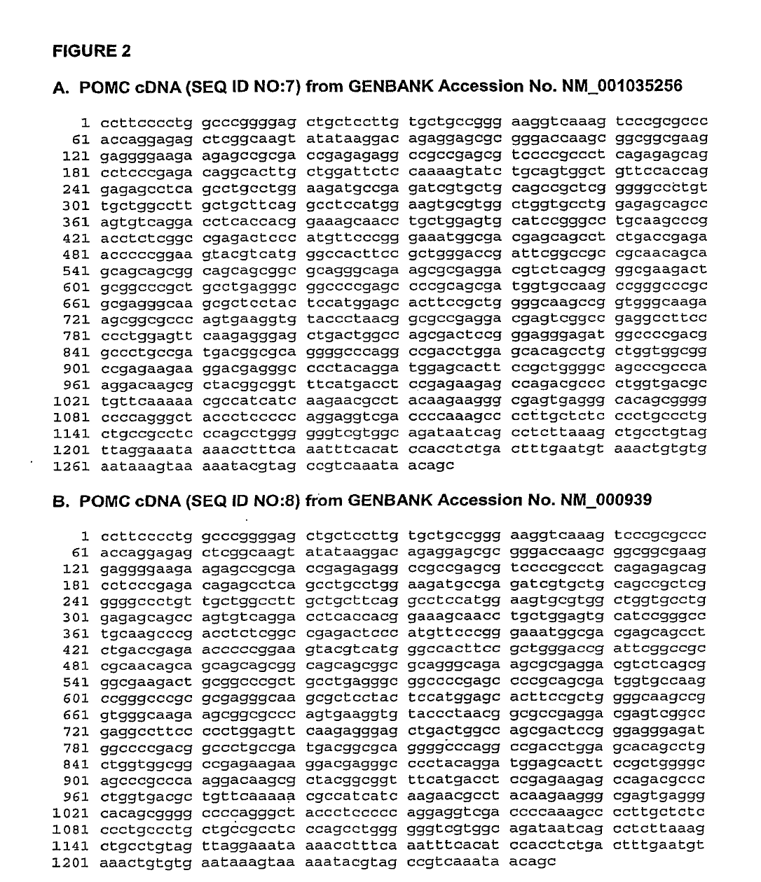

FIG. 3 shows the amino acid sequence of envelope (Env) gp160 (SEQ ID NO:9) of the SF2 isolate of HIV-1. Residues 1-27 correspond to the signal peptide sequence, residues 28-509 correspond to the gp120 sequence (SEQ ID NO:15), and residues 510-855 correspond to the gp41 sequence (SEQ ID NO:16).

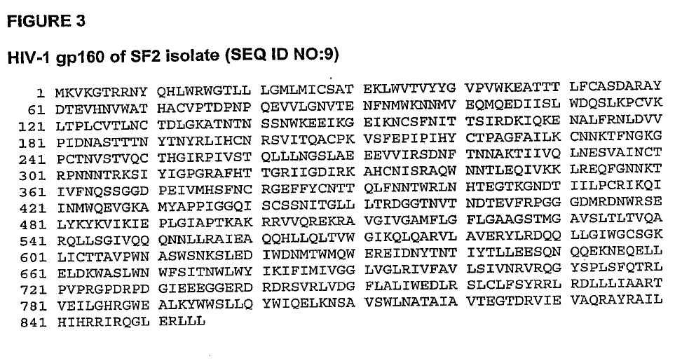

FIG. 4 shows the cDNA sequence (SEQ ID NO:10) encoding the Env gp160 sequence of the previous figure. This sequence corresponds to the HIV-1 SF2 isolate of GENBANK Accession No. KO2007 (Sanchez-Pescador et al., Science, 227:484-492, 1985).

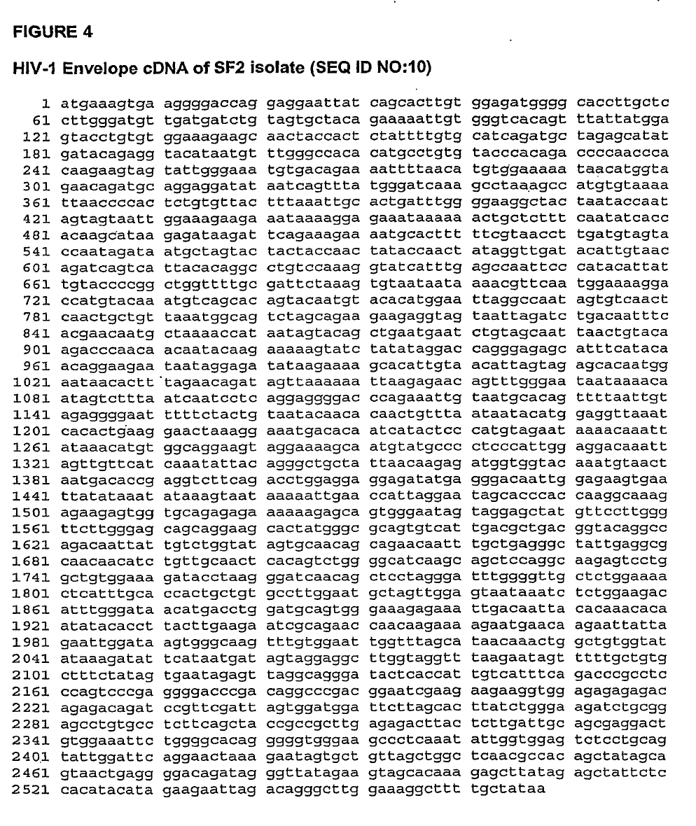

FIG. 5A shows the amino acid sequence of the melanocortin 4 receptor (MC4R as set forth as SEQ ID NO:11). FIG. 5B shows the cDNA sequence of MC4R (SEQ ID NO:12) from GENBANK Accession No. NM_{—}005912.

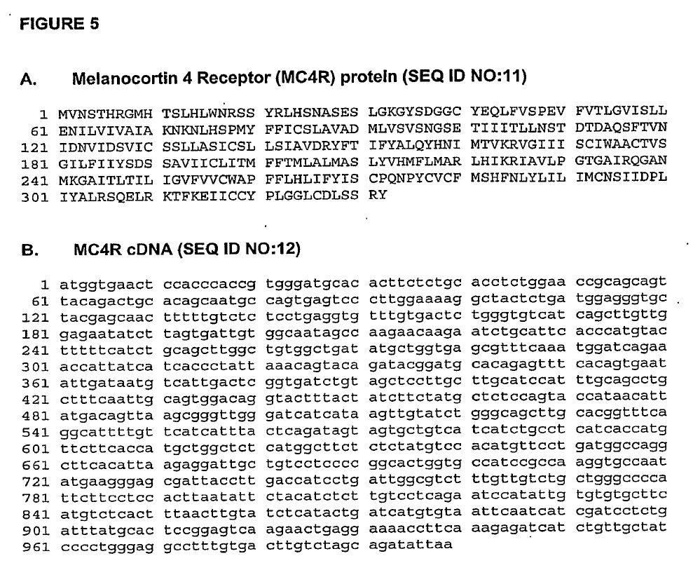

FIG. 6A shows the amino acid sequence of the melanocortin 5 receptor (MC5R as set forth as SEQ ID NO:13). FIG. 5B shows the cDNA sequence of MC5R (SEQ ID NO:14) from GENBANK Accession No. NM_{—}005913.

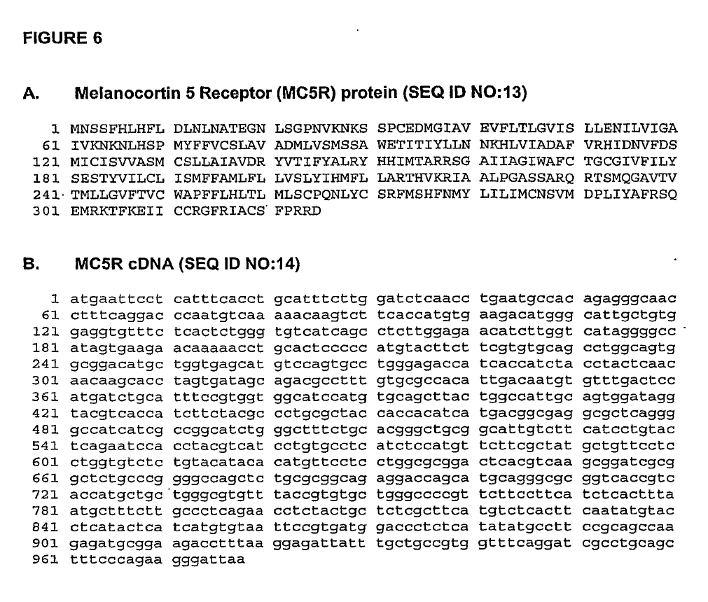

FIG. 7 provides an alignment of the melanocortin receptor binding peptide of the HIV-1SF2 gp120 (SEQ ID NO:17) with the surface glycoproteins of HIV-2CAM2 (SEQ ID NO:18), HIV-2D205 (SEQ ID NO:19), HIV-2D194 (SEQ ID NO:20) and SIVmac251 (SEQ ID NO:21). Note that HIV-1 gp120, but not the HIV-2 and SIV surface glycoproteins, contain the tryptophan residue conserved among the POMC-derived hormones.

FIG. 8 demonstrates that the HIV-1SF2 #79 termed the G25G peptide (SEQ ID NO:17) activates the human MC4R.

FIG. 9 demonstrates that the HIV-1SF2 #33 termed the K13M (SEQ ID NO:6) synthesized in the reverse orientation also activates the human MC4R.

FIG. 10 illustrates the dysregulation of CD4+CD25+ regulatory T cell development mediated by HIV-1 gp120 stimulation of MC5R+ T cells, leading to hyperactivation and subsequent T cell depletion. The sequence of the unbound alpha-MSH is provided as SEQ ID NO:2, while the sequence of the bound K13M viral melanotropin epitope is provided as SEQ ID NO:6.

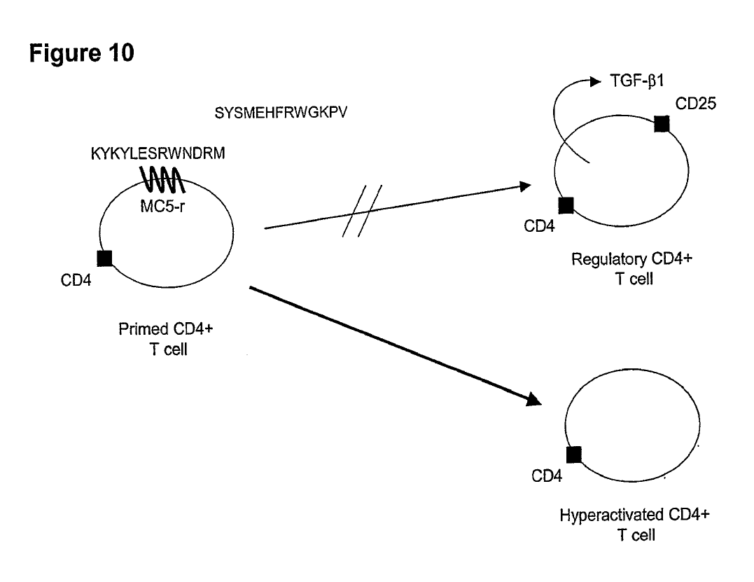

FIG. 11 illustrates a structure of HIV-1 gp120 on the left and a structure of a 50 kDa fragment on the right. HIV-1 gp120 is cleaved by thrombin within the V3 loop liberating two fragments (approximately 70 kDa and 50 kDa). The location of an HIV-1 melanotropin epitope, which is exposed upon cleavage is indicated by the arrow.

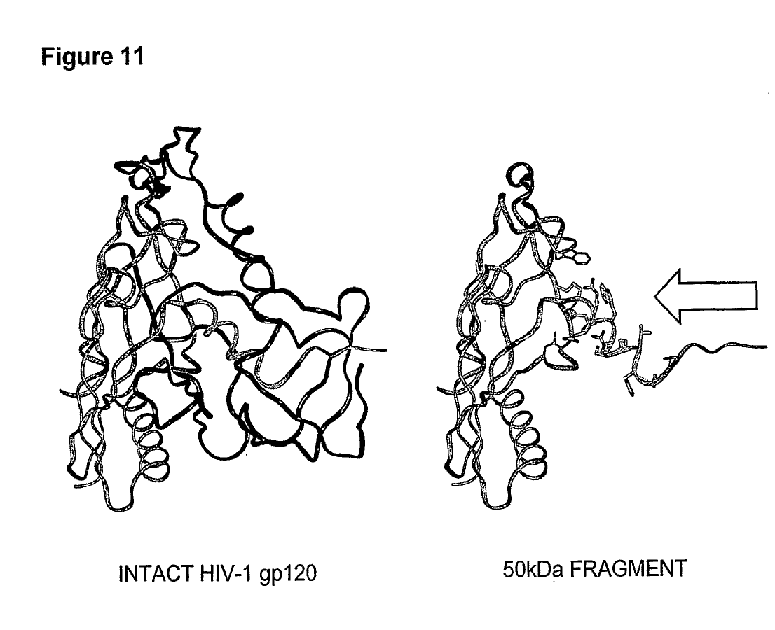

Table 1 lists the pharmacological properties and distribution of melanocortin receptor subtypes.

Table 2 lists characteristics of participants in the UCSF SCOPE cohort.

Table 3 lists the human central nervous system molecules of the National Institute for Mental Health (NIMH) Psychoactive Drug Screening Program (PDSP), which are screened in binding and/or functional assays with HIV-1 gp120, its degradation products and synthetic peptides comprising an HIV-I melanotropin epitope.

TABLE 1
Pharmacological properties and distribution of melanocortin receptor subtypes

| Subtype | Ligand potency/endogenous antagonists | Tissue distribution | Functions |
|---|---|---|---|
| MC1R | alpha-MSH = ACTH > beta-MSH >> gamma-MSH | Macrophages, leukocytes | Anti-inflammation |
| | Agouti | Melanocytes, keratinocytes | Pigmentation |
| MC2R | ACTH | Adrenal cortex | Steroidogenesis |
| MC3R | alpha-MSH = ACTH = beta-MSH = gamma-MSH | CNS, placenta | Autonomic functions |
| | AGRP | | Energy homeostasis |
| | | | Anti-inflammation |
| MC4R | alpha-MSH = ACTH = beta-MSH >> gamma-MSH | CNS | Feeding and energy homeostasis |
| | Agouti, AGRP | | Regulation of HPA axis |
| | | | Anti-inflammation |
| | | | Erectile activity |
| | | | Nociception |
| MC5R | alpha-MSH > ACTH > beta-MSH > gamma-MSH | Ubiquitous in peripheral | Regulation of exocrine secretion |
| | | tissues | |
| | | T cells | Induction of CD25+ |
| | | | CD4+ regulatory T cells |

Muceniece R, et al., beta-MSH inhibits brain inflammation via MC3/4 receptors and NF-kB signaling. J Neuroimmunology 2005; 169: 13-19.
Chaki S., Okuyarna S. Involvement of melanocortin-4 receptor in anxiety and depression. Peptides 2005; 26: 1952-1964.

TABLE 2

| | [symbol] | | [symbol] | | [symbol] | | [symbol] | | [symbol] | |
|---|---|---|---|---|---|---|---|---|---|---|
| Age, median (IQR) | 43 | (36-47) | 45 | (39-50) | 45 | (40-51) | 45 | (38-50) | 44 | (39-50) |
| Male Gender (%) | 81 | (73.6) | 107 | (90.7) | 281 | (90.1) | 74 | (79.6) | 534 | (85.8) |
| Ethnicity (%) | | | | | | | | | | |
| White | 59 | (53.6) | 74 | (62.7) | 173 | (55.5) | 42 | (44.2) | 348 | (54.8) |
| African American | 33 | (30.0) | 19 | (16.1) | 70 | (22.4) | 35 | (36.8) | 157 | (24.7) |
| Hispanic | 7 | (6.4) | 8 | (6.8) | 37 | (11.9) | 8 | (8.4) | 60 | (9.5) |
| Asian/PI | 6 | (5.5) | 8 | (6.8) | 8 | (2.6) | 2 | (2.1) | 24 | (3.8) |
| Other | 5 | (4.6) | 9 | (7.6) | 24 | (7.7) | 8 | (8.4) | 46 | (7.2) |
| Sexual Orientation | | | | | | | | | | |
| (%) | | | | | | | | | | |
| Heterosexual | 40 | (36.4) | 20 | (17.0) | 57 | (18.3) | 29 | (30.5) | 146 | (23.0) |
| Homo/Bisexual | 70 | (63.6) | 98 | (83.0) | 254 | (81.7) | 66 | (69.5) | 488 | (77.0) |
| CD4, median (IQR) | 298 | (192-413) | 472 | (338-665) | 250 | (131-437) | 663 | (471-781) | 360 | (197-560) |
| Plasma HIV RNA | | | | | | | | | | |
| (%) | | | | | | | | | | |
| <75 copies/ml | 0 | | 112 | (95.7) | 39 | (12.9) | 29 | (36.3) | 180 | (29.6) |
| 75-1,000 | 0 | | 4 | (3.4) | 61 | (20.2) | 25 | (31.3) | 90 | (14.8) |
| 1001-10,000 | 27 | (24.5) | 1 | (0.9) | 76 | (25.2) | 24 | (30.0) | 128 | (21.0) |
| 10,001-100,000 | 52 | (47.3) | 0 | | 90 | (29.8) | 2 | (2.5) | 144 | (23.7) |
| >100,000 | 31 | (28.2) | 0 | | 36 | (11.9) | 0 | | 67 | (11.0) |

Characteristics of participants in the UCSF SCOPE cohort. [symbol]
indicates data missing or illegible when filed

TABLE 3
National Institutes of Mental Health (NIMH)
Psychoactive Drug Screening Program (PDSP)
Binding Assay Components

| | | |
|---|---|---|
| RECEPTOR | RADIOLIGAND | REFERENCE COMPOUND |

5-HT Receptors

| | | |
|---|---|---|
| 5-HT1A | 3H-8-OH-DPAT | 5-HT |
| 5-HT1B | 3H-GR125743 | ERGOTAMINE |
| 5-HT1Da | 3H-GR125743 | ERGOTAMINE |
| 5-HT1Db | 3H-GR125743 | ERGOTAMINE |
| 5-HT1E | 3H-5HT | 5HT |
| 5-HT1F | 3H-5HT | 5HT |
| 5-HT2A | 3H-ketanserin | Chlorpromazine |
| 5-HT2B | 3H-LSD | Norphenflueramine |
| 5-HT2C | 3H-mesulergine | Chlorpromazine |
| 5-HT3 | 3H-zacopride | LY-278,584 |
| 5-HT4 | 3H-5HT | SDZ-205557 |
| 5-HT5A | 3H-LSD | Ergotamine |
| 5-HT6 | 3H-LSD | Chlorpromazine |
| 5-HT7 | 3H-LSD | Chlorpromazine |

Dopamine Receptors

| | | |
|---|---|---|
| D1 | 3H-SCH23390 | SKF 38393 |
| D2 | 3H-N-Methylspiperone | HALOPERIDOL |
| D3 | 3H-N-Methylspiperone | Chlorpromazine |
| D4 | 3H-N-Methylspiperone | Chlorpromazine |
| D5 | SKF38393 | SCH23390 |

Muscarinic Receptors

| | | |
|---|---|---|
| M1 | 3H-QNB | Pirenzepine |
| M2 | 3H-QNB | Methoctramine |
| M3 | 3H-QNB | 4-DAMP |
| M4 | 3H-QNB | Tropicamide |
| M5 | 3H-QNB | Pirenzepine |

Adrenergic Receptors

| | | |
|---|---|---|
| Alpha_{1A} | 3H-Prazosin | uripadil |
| Alpha_{1B} | 3H-Prazosin | corynathine |
| Alpha_{2A} | 3H-Cholidine | oxymetazoline |
| Alpha_{2B} | 3H-Cholidine | prazosin |
| Alpha_{2C} | 3H-Cholidine | prazosin |
| Beta₁ | 125I-Pindolol | Atenolol |
| Beta₂ | 125I-Pindolol | Atenolol |
| Beta₃ | 125I-Pindolol | Atenolol |

Opiate Receptors

| | | |
|---|---|---|
| Mu | 3H-diprenorphine | Naloxone |
| Delta | 3H-DADLE | Naltrindole |
| Kappa | 3H-Bremazocine | Naloxone |

Nicotinic Receptors

| | | |
|---|---|---|
| many subtypes | 3H-Epibatidine | Nicotine |

GABA Receptors

| | | |
|---|---|---|
| GABA | 3H-Muscimol | GABA |
| GABA | 3H-SR 95531 | GABA |
| GABA | 3H-Ro 15-4513 | Benzodiazepine |
| GABA | 3H-Ro 15-1788 | Benzodiazepine |
| GABA | 35S-TBPS | Receptor channel |

Ionotropic Glutamate Receptors

| | | |
|---|---|---|
| NMDA | 3H-MK801 | MK-801 |
| PCP site | 3H-TCP | PCP |
| Kainate site | 3H-Kainic acid | Glutamic acid |

Peptide Receptors

| | | |
|---|---|---|
| Galanin | 125I-GALANIN | Galanin |
| CRFR1 | 125I-CRF | CRF |
| CRFR2 | | |
| NPY-Y1 | 125I-NPY | L³¹,P³⁴ NPY |
| NPY-Y2 | 125I-NPY | NPY 3-36 |
| Neurotensin | 125I-Tyr³-NT | Neurotensin |

Transporters

| | | |
|---|---|---|
| VMAT2 | 3H-ketanserin | Reserpine |
| SERT | 3H-Citalopram | Fluoxetine |
| NET | 3H-nisoxitine | Nortriptyline |
| DAT | 3H-WIN35428 | 4′,4″-Diflouro-3a- |
| | | (diphenylmethoxy)tropane |
| | | HCL |

### Definitions

The term “gene” refers to a nucleic acid (e.g., DNA) sequence that comprises coding sequences necessary for the production of a polypeptide or precursor (e.g., HIV-1 ENV). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions”0 or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In particular, the term “HIV-1 ENV gene” refers to the full-length HIV-1 ENV nucleotide sequence. However, it is also intended that the term encompass fragments of the HIV-1 ENV sequence, as well as other domains within the full-length HIV-1 ENV nucleotide sequence. Furthermore, the terms “HIV-1 ENV nucleotide sequence” or “HIV-1 ENV polynucleotide sequence” encompasses DNA, cDNA, and RNA (e.g. mRNA) sequences.

Where amino acid sequence is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified”, “mutant”, and “variant” refer to a gene or gene product that displays modifications in sequence and or functional properties (i.e., altered characteristics) when compared to the wild-type gene or gene product. It is noted that naturally occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring, and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

As used herein, the term “competes for binding” is used in reference to a first polypeptide with an activity which binds to the same substrate as does a second polypeptide with an activity, where the second polypeptide is a variant of the first polypeptide, or a related or dissimilar polypeptide. The efficiency (e.g., kinetics or thermodynamics) of binding by the first polypeptide may be the same as or greater than or less than the efficiency substrate binding by the second polypeptide. For example, the equilibrium binding constant (K_{D}) for binding to the substrate may be different for the two polypeptides. The term “Kₘ” as used herein refers to the Michaelis-Menton constant for an enzyme and is defined as the concentration of the specific substrate at which a given enzyme yields one-half its maximum velocity in an enzyme catalyzed reaction.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization, 1985). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5× SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1× SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5× SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0× SSPE, 1.0% SDS at 42° C. when a probe of about 500 nucleotides in length is employed.

“Low stringency conditions” comprise conditions equivalent to binding or hybridization at 42° C. in a solution consisting of 5× SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)) and 100 g/ml denatured salmon sperm DNA followed by washing in a solution comprising 5× SSPE, 0.1% SDS at 42° C. when a probe of about 500 nucleotides in length is employed.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv Appl Math, 2: 482 (1981)] by the homology alignment algorithm of Needleman and Wunsch [Needleman and Wunsch, J Mol Biol, 48:443 (1970)], by the search for similarity method of Pearson and Lipman [Pearson and Lipman, Proc Natl Acad Sci, USA, 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 80 percent sequence identity, preferably at least 90, 95, or 97 percent sequence identity and more preferably at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention (e.g., HIV-1 Env)

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95, 97 or 99 percent sequence identity. Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 10 amino acids long, more preferably at least 15, amino acids long, and most preferably 20 amino acids long or longer (e.g., 25 to 50 amino acids long), and span the portion of the polypeptide (e.g., K13M of SEQ ID NO:6 or G25G of SEQ ID NO:17) required for intermolecular binding of the compositions (claimed in the present invention) with its various ligands and/or substrates.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally occurring.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be isolated sorted from other nucleic acids. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences.

As used herein, the term “target,” when used in reference to the polymerase chain reaction, refers to the region of nucleic acid bounded by the primers used for polymerase chain reaction.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are usefull in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “polymerase chain reaction” (“PCR”) refers to the method of Mullis (U.S. Pat. Nos. 4,683,195, 4,683,202, and 4,965,188, hereby incorporated by reference) for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., mRNA). Included within this definition are antisense RNA (“as RNA”) molecules involved in gene regulation by bacteria. Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand. Regions of a nucleic acid sequences that are accessible to antisense molecules can be determined using available computer analysis methods.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding MC4R includes, by way of example, such nucleic acid in cells ordinarily expressing MC4R where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets that specify stop codons (i.e., TAA, TAG, TGA).

As used herein, the term “purified” or “to purify” refers to the removal of contaminants from a sample. For example, HIV-1 Env antibodies are purified by removal of contaminating non-immunoglobulin proteins; they are also purified by the removal of immunoglobulin that does not bind HIV-1 Env. The removal of non-immunoglobulin proteins and/or the removal of immunoglobulins that do not bind HIV-1 Env results in an increase in the percent of HIV-1 Env-reactive immunoglobulins in the sample. In another example, recombinant HIV-1 Env polypeptides are expressed in bacterial host cells and the polypeptides are purified by the removal of host cell proteins; the percent of recombinant HIV-1 Env polypeptides is thereby increased in the sample.

The term “recombinant DNA” refers to a DNA molecule that is comprised of segments of DNA joined together by means of molecular biology techniques. Similarly, the term “recombinant protein” refers to a protein molecule that is expressed from recombinant DNA.

The term “fusion protein” as used herein refers to a protein formed by expression of a hybrid gene made by combining two gene sequences. Typically this is accomplished by cloning a cDNA into an expression vector in frame with an existing gene. The fusion partner may act as a reporter (e.g., βgal), may provide a tool for isolation purposes (e.g., GST) or may increase the half-life of the protein in vivo (e.g., IgG Fc).

Suitable systems for production of recombinant proteins include but are not limited to prokaryotic (e.g., Escherichia coli), yeast (e.g. Saccaromyces cerevisiae), insect (e.g., baculovirus), mammalian (e.g., Chinese hamster ovary), plant (e.g. safflower), and cell-free systems (e.g., rabbit reticulocyte).

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58, 1989).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp 7.39-7.52, 1989).

The terms “Western blot,” “Western immunoblot” “immunoblot” and “Western” refer to the immunological analysis of protein(s), polypeptides or peptides that have been immobilized onto a membrane support. The proteins are first resolved by polyacrylamide gel electrophoresis (i.e., SDS-PAGE) to separate the proteins, followed by transfer of the protein from the gel to a solid support, such as nitrocellulose or a nylon membrane. The immobilized proteins are then exposed to an antibody having reactivity towards an antigen of interest. The binding of the antibody (i.e., the primary antibody) is detected by use of a secondary antibody that specifically binds the primary antibody. The secondary antibody is typically conjugated to an enzyme that permits visualization of the antigen-antibody complex by the production of a colored reaction product or catalyzes a luminescent enzymatic reaction (e.g., the ECL reagent, Amersham). In other embodiments, the binding of the primary antibodies maybe detected by the use of radiolabeled secondary antibodies.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antibody” refers to polyclonal and monoclonal antibodies. Polyclonal antibodies which are formed in the animal as the result of an immunological reaction against a protein of interest or a fragment thereof, can then be readily isolated from the blood using well-known methods and purified by column chromatography, for example. Monoclonal antibodies can also be prepared using known methods (See, e.g., Winter and Milstein, Nature, 349, 293-299, 1991). As used herein, the term “antibody” encompasses recombinantly prepared, and modified antibodies and antigen-binding fragments thereof, such as chimeric antibodies, humanized antibodies, multifunctional antibodies, bispecific or oligo-specific antibodies, single-stranded antibodies and F(ab) or F(ab)₂ fragments. The term “reactive” in used in reference to an antibody indicates that the antibody is capable of binding an antigen of interest. For example, an HIV-1 melanotropin epitope-reactive antibody is an antibody that binds to HIV-1 gp120, or to a fragment of HIV-1 gp120 (e.g., a peptide comprising the amino acid sequence of SEQ ID NO:17).

As used herein, the term “immunoassay” refers to any assay that uses at least one specific antibody for the detection or quantitation of an antigen. Immunoassays include, but are not limited to, Western blots, ELISAs, radioimmunoassays, and immunofluorescence assays. Many different ELISA formats are known in the art, any of which will find use in the present invention. However, it is not intended that the present invention be limited to these assays.

As used herein, the term “ELISA” refers to enzyme-linked immunosorbent assay (or EIA). Numerous ELISA methods and applications are known in the art, and are described in many references (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J., 1998; Harlow and Lane (eds.), Antibodies: A Laboratory Manual, Cold Spring Harbor Laboratory Press, 1988; Ausubel et al. (eds.), Current Protocols in Molecular Biology, Ch. 11, John Wiley & Sons, Inc., New York, 1994). In addition, there are numerous commercially available ELISA test systems.

As used herein, a “detection antibody” is an antibody that carries a means for visualization or quantitation, which is typically a conjugated enzyme moiety that typically yields a colored or fluorescent reaction product following the addition of a suitable substrate. Conjugated enzymes commonly used with detection antibodies in the ELISA include horseradish peroxidase, urease, alkaline phosphatase, glucoamylase and β-galactosidase. In some embodiments, the detection antibody is directed against the antigen of interest, while in other embodiments, the detection antibody is an anti-species antibody. Alternatively, the detection antibody is prepared with a label such as biotin, a fluorescent marker, or a radioisotope, and is detected and/or quantitated using this label.

As used herein, the terms “reporter reagent,” “reporter molecule,” “detection substrate” and “detection reagent” are used in reference to reagents that permit the detection and/or quantitation of an antibody bound to an antigen. For example, in some embodiments, the reporter reagent is a calorimetric substrate for an enzyme that has been conjugated to an antibody. Addition of a suitable substrate to the antibody-enzyme conjugate results in the production of a calorimetric or fluorimetric signal (e.g., following the binding of the conjugated antibody to the antigen of interest). Other reporter reagents include, but are not limited to, radioactive compounds. This definition also encompasses the use of biotin and avidin-based compounds (e.g., including but not limited to neutravidin and streptavidin) as part of the detection system.

The term “transgene” as used herein refers to a foreign gene that is placed into an organism by introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside at the same location as does the naturally occurring gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term host cell refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol, 52:456, 1973), has been modified by several groups to optimize conditions for particular types of cells. The art is well aware of these numerous modifications.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The terms “patient” and “subject” refer to a mammal (human or animal) that is a candidate for receiving medical treatment.

The term “control” refers to subjects or samples which provide a basis for comparison for experimental subjects or samples. For instance, the use of control subjects or samples permits determinations to be made regarding the efficacy of experimental procedures. In some embodiments, the term “control subject” refers to animals that receive a mock treatment (e.g., PBS alone or normal rabbit IgG in saline).

The terms “sample” and “specimen” are used in their broadest sense. On the one hand, they are meant to include a specimen or culture. On the other hand, they are meant to include both biological and environmental samples. These terms encompasses all types of samples obtained from humans and other animals, including but not limited to, body fluids such as urine, blood (e.g., including sera or plasma), fecal matter, cerebrospinal fluid, semen, saliva, and wound exudates, as well as solid tissue. However, these examples are not to be construed as limiting the sample types applicable to the present invention.

The term “leukocyte” as used herein, refers to cells called white blood cells that help the body fight infections and other diseases, and include for instance granulocytes (e.g., neutrophils, eosinophils, basophils), mononuclear phagocytes, and lymphocytes (e.g., B cells, T cells, natural killer cells).

As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

As used herein, the term “agonist” refers to molecules or compounds that mimic the action of a “native” or “natural” compound. Agonists may be homologous to these natural compounds in respect to conformation, charge or other characteristics. Thus, agonists may be recognized by receptors expressed on cell surfaces. This recognition may result in physiologic and/or biochemical changes within the cell, such that the cell reacts to the presence of the agonist in the same manner as if the natural compound was present. Agonists may include proteins, nucleic acids, carbohydrates, or any other molecules that bind or interact with a melanocortin receptor.

As used herein, the terms “antagonist” and “inhibitor” refer to molecules or compounds that inhibit the action of a “native” or “natural” compound. Antagonists may or may not be homologous to these natural compounds in respect to conformation, charge or other characteristics. Thus, antagonists may be recognized by the same or different receptors that are recognized by an agonist. Antagonists may have allosteric effects, which prevent the action of an agonist (e.g., prevent HIV-1 melanotropin epitope or HIV-1 gp120 from binding to a melanocortin receptor). In contrast to the agonists, antagonistic compounds do not result in physiologic and/or biochemical changes within the cell such that the cell reacts to the presence of the antagonist in the same manner as if the natural compound was present. Antagonists and inhibitors may include proteins, nucleic acids, carbohydrates, or any other molecules that bind or interact with melanocortin receptors or a polypeptide comprising an HIV-1 melanotropin epitope or which prevent stimulation of a melanocortin receptor by a polypeptide comprising an HIV-1 melanotropin epitope.

As used herein, the term “providing a prognosis” refers to providing information regarding the impact of the presence of HIV-1-infection on a subject's future health (e.g., includes a determination of the likelihood or relative risk of developing one or more of HIV-1-related cachexia, wasting disease, T regulatory cell dysfunction and CNS disease, in comparison to other HIV-1-infected individuals).

As used herein, the term “instructions for using said kit” includes instructions for using the reagents contained in the kit for the detection of pathogenic (e.g., HIV-1) melanotropin epitope-reactive antibodies in a sample from a subject. In some embodiments, the instructions further comprise the statement of intended use required by the U.S. Food and Drug Administration (FDA) in labeling in vitro diagnostic products. The FDA classifies in vitro diagnostics as medical devices and requires that they be approved through the 510(k) or analyte specific reagent (ASR) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use. Where applicable, photographs or engineering drawings should be supplied; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; 8) Any additional information regarding the in vitro diagnostic product requested that is necessary for the FDA to make a substantial equivalency determination. Additional information is available at the Internet web page of the U.S. FDA.

### DETAILED DESCRIPTION OF THE INVENTION

The present invention relates to aberrant cell signaling by the HIV type I envelope glycoprotein. In particular, the present invention provides compositions and methods for identification of inhibitors of melanocortin receptor pathway stimulation by HIV-1 gp120 and its degradation products. The inhibitors so identified are contemplated to be suitable for treating HIV-related cachexia, T cell hyperactivation and central nervous system disease.

### I. The Melanocortin System

Melanocortins are a group of pro-opiomelanocortin (POMC) derived peptides that are comprised of adrenocorticotrophin (ACTH) and alpha-, beta-, and gamma-melanocyte-stimulating hormones (MSH) among other peptide hormones (See, e.g., Raffin-Sanson et al., Eur J Endocrinol, 149:79-90, 2003). These bioactive peptides act as endogenous agonists for cell surface melanocortin receptors and play a variety of physiological roles in steroidogenesis, pigmentation, anti-inflammation, control of food intake, energy expenditure, and sexual behavior. Molecular cloning studies have revealed the existence of five subtypes of melanocortin receptors termed MC1R, MC2R, MC3R, MC4R, and MC5R, which form a distinct family of G protein coupled receptors. All five melanocortin receptors activate adenylyl cyclases via stimulatory G proteins, thereby elevating intracellular cAMP (Cone, Nature Neuroscience, 8:571-578, 2005). The five subtypes of receptors are distinguishable by their tissue distribution, physiological function, and their ability to recognize various melanocortin peptides (Table 1).

The melanocortin-4 receptor (MC4R) is expressed in multiple central nervous system (CNS) sites and has been shown to be critical in maintaining body weight homeostasis (O'Rahilly et al., Nature Medicine, 10:351-352, 2004; and Huszar et al., Cell, 88:131-141, 1997). Activation of melanocortin-4 receptors reduces body fat stores by decreasing food intake and increasing energy expenditure (Balthasar et al., Cell, 123:493-505, 2005). Recent studies have revealed evidence for a direct central modulation of sympathetic outflow to white adipose tissue through melanocortin-4 receptors resulting in lipid mobilization and decreased adiposity (Berthound et al., Am J Physiol Regul Integr Comp Physiol, 289:1236-1237,2005). The significance of the melanocortin-4 receptor in energy homeostasis in humans is demonstrated by the fact that mutations in the melanocortin-4 receptor are now recognized as the most common form of monogenic obesity in humans (Srinivasan et al., J Clin Invest, 114:1158-1164; O'Rahilly et al., Nature Medicine, 10:351-352, 2004; and Farooqi et al., N Engl J Med, 348:1085-1095, 2003).

### II. HIV-1 Envelope Glycoproteins

The envelope (Env) glycoproteins of HIV mediate viral attachment to and subsequent membrane fusion with host cells (Wyatt and Sodroski, Science, 280:1884-1888, 1998). The gp160 Env glycoproteins assemble as trimers during synthesis and are cleaved during transport to the infected cell surface into ectodomain (gp120) and transmembrane (gp41) fragments. Cleavage permits Env to undergo conformational changes upon binding to the CD4 receptor (Dalgleish et al., Nature, 312, 763-767, 1984; and Klatzmann et al., Nature, 312:767-768, 1984) and the CXCR4 or CCR5 co-receptor (Feng et al., Science, 272:872-877, 1996; Trkola et al., Nature, 384:184-187, 1996; and Wu et al., Nature, 384:179-183, 1996). Comparison of gp120 amino acid sequences from multiple viral isolates led to the identification of five variable regions flanked by five conserved regions (Starcich et al., Cell, 45:637-648, 1986). The conserved regions of the gp120 ectodomain form structures important for interaction with the gp41 transmembrane domain and for interaction with the viral receptors on host cells.

Crystal structures of HIV gp120 have been determined (See, e.g., Berger, Nature Structure Biology, 5:671-674; Kwong et al., Nature, 393:648-659,1998; Moore and Binley, Nature, 393:630-631, 1998; Rizzuto et al., Science, 280:1949-1953; Wyatt and Sodroski, Science, 280:1884-1888, 1998; and Wyatt et al., Nature, 393:705-711, 1998) shedding light on the relationship between envelope structure and function. The gp120 glycoprotein is shed from the envelope complex and can be found in the serum of AIDS patients at concentrations of ˜12-92 ng/ml (Schneider et al., J Gen Virol, 67:2533-2538, 1986; and Oh et al., J AIDS, 5:251-256, 1992). During natural infection, gp120 elicits both virus-neutralizing and non-neutralizing antibodies. Non-neutralizing antibodies are frequently directed against regions of gp120 that are exposed in soluble gp120 but inaccessible in the trimeric envelope complex. In contrast, neutralizing antibodies recognize epitopes in the trimeric envelope complex, typically in regions of gp120 adjacent to receptor-binding sites (Sattentau and Moore, J Exp Med, 182:185-196, 1995; and Moore and Sodroski, J Virol, 70:1863-1872, 1996).

HIV-1 gp120 was shown to be susceptible to V3 loop cleavage yielding two fragments of 70 kDa and 50 kDa (Werner and Levy, J Virol, 67:2566-2574, 1993). In particular, thrombin and tryptase were found to cleave gp120 at a tryptic site (GPGR/AFVT set forth as SEQ ID NO:32), while cathepsin E was found to cleave gp120 at chymotrypsin-like site (GPGRAF/VT set forth as SEQ ID NO:32) in studies using HIV-1 gp120 corresponding to the IIIB isolate (Clements et al., AIDS Res Hum Retroviruses, 7:3-16, 1991). It has also been suggested that a thrombin-like protease cleaves gp120 at (GP/GRAFVT set forth as SEQ ID NO:32) after a CD4 binding induced conformational change (Johnson et al., FEBS Lett, 337:4-8, 1994). Since then a T-cell membrane associate serine protease, tryptase TL2 was found to cleave gp120 within the V3 loop (Niwa et al., Eur J Biochem, 237:64-70, 1996). The present invention contemplates that the one or both gp120 degradation fragments (e.g., 70 kDa amino terminal fragment and/or 50 kDa carboxy terminal fragment) play a role in HIV-1 related pathology.

III. HIV-1 gp120 Mediates AIDS Pathogenesis

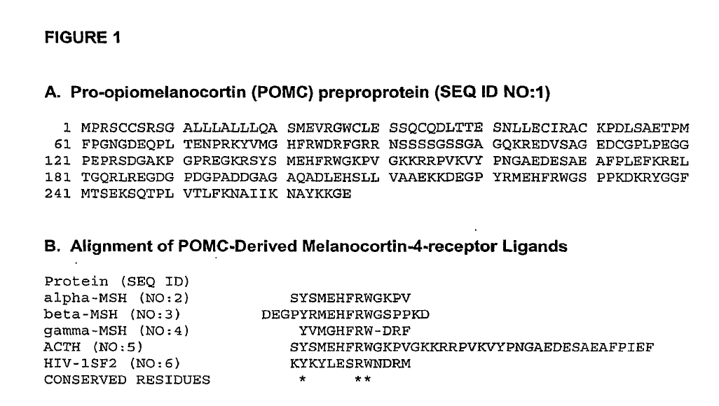

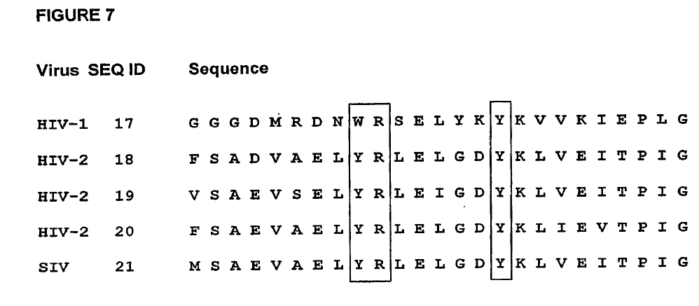

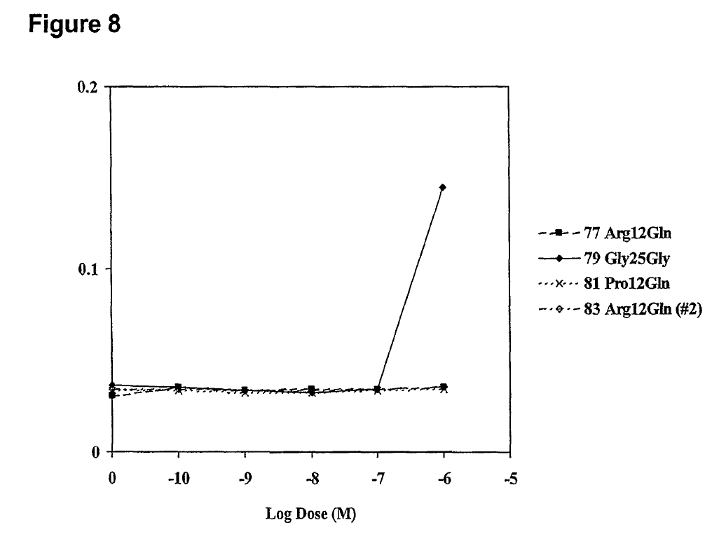

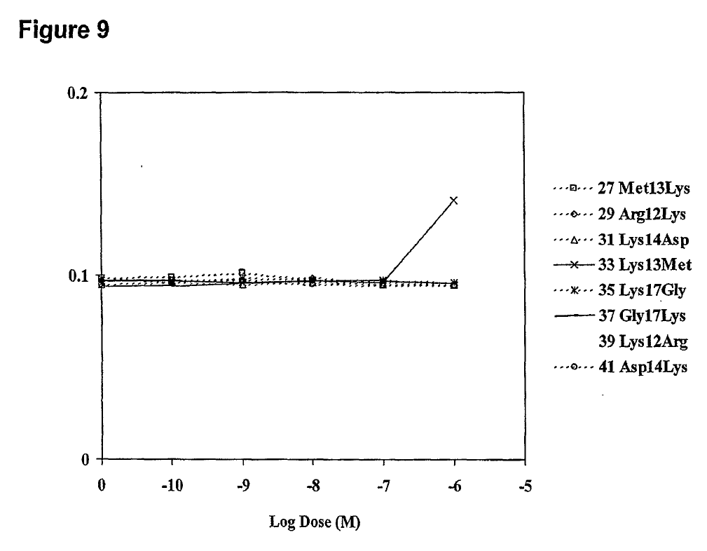

Conformational mimicry of host peptides by HIV-1 gp120 has been postulated to play a role in various aspects of AIDS pathogenesis. In fact, prior to development of the present invention, the inventor had proposed that peptide T, a fragment of the second variable region of gp120 (ASTTTNYT, corresponding to residues 185-192 of the HIV-1SF2 sequence, set forth as SEQ ID NO:22), mimicked the gamma-MSH hormone (Dominy, Med Hypothesis, 27:1-4, 1988). Although this hypothesis was not substantiated, further analysis of the HIV-1 gp120 amino acid sequence has revealed the existence of a melanotropin epitope near the carboxy-terminus of HIV-1 gp120, as described herein in Example 1. Specifically, during development of the present invention, a peptide termed G25G (SEQ ID NO:17) of the fifth conserved domain of HIV-1 gp120 including residues within the fifth alpha helix and the twenty fifth beta sheet, was found to be a weak agonist of the MC4R (FIGS. 7 and 8). In addition, as described herein for the first time, a peptide termed K13M (SEQ ID NO:6) synthesized in the reverse orientation of sequence within the G25G peptide was found to share critical tryptophan, arginine and tyrosine residues with alpha-MSH, beta-MSH, gamma-MSH and ACTH (FIG. 1). Importantly, the K13M peptide was also found to be a weak agonist of the MC4R (FIG. 9).

A. AIDS Wasting Syndrome

The present invention contemplates that the melanotropin epitope in HIV-1 gp120 or its degradation products binds to and activates MC4R or other CNS cell surface molecules in vivo, thereby playing roles in the development of HIV-related cachexia and AIDS wasting syndrome. In particular, it is contemplated that chronic stimulation of MC4R by HIV-1 gp120 contributes to anorexia and increased energy expenditure. Recent studies, when viewed in the context of the present invention, provide support for the present invention. Specifically, HIV-1 gp120 was found to be present in the central nervous system during all stages of disease, including in patients with HIV-1-associated dementia (Ritola et al., J Virol, 79:10830-10834, 2005). Moreover, intracerebroventricular administration of HIV-1IIIB gp120 daily for five days, significantly decreased food and water intake by rats (Guzman et al., Neurosci Lett, 396:50-53, 2006), resulting in decreased weight gain in comparison to control rats receiving vehicle alone.

Although, knowledge of the mechanism(s) is not required to make and use the present invention and the present invention is not limited to any particular mechanism of action, the spontaneous shedding of gp120 by HIV-1-infected cells is contemplated to resemble an exocrine gland secreting a peptide hormone. In an extension of this analogy, the primary endogenous ligand for MC4R, alpha-MSH, is physiologically active at a concentration of 30 pg/ml, whereas the circulating concentration of HIV-1 gp120 in an AIDS patient is on the order of 12-92 ng/ml (e.g., 3 orders of magnitude greater). Thus, supraphysiological concentration of gp120 is contemplated to compete with neuropeptide hormones for binding to the MC4R.

B. Regulatory T Cell Dysfunction

In addition to playing a critical role in body weight homeostasis, the melanocortin system is crucial in regulating the level of T cell activation. Through the melanocortin-5 receptor (MC5-R) expressed on the surface of antigen-primed T cells alpha-MSH stimulates development of CD4+CD25+ regulatory T (Treg) cells. CD4+CD25+ regulatory T cells in turn suppress activation of other T cell subsets in part through the release of transforming growth factor (TGF)-beta (Taylor and Namba, Immunol Cell Biol, 79:358-367, 2001). More recently high levels of MC1R, MC2R and MC3R mRNAs, as well as MC5R mRNA have been found in CD4+ T cells, while moderate levels of these mRNAs were found in NK cells, monocytes and granulocytes (Anderson et al., Scand J Immunol, 61:279-284, 2005). The present invention contemplates that HIV-1 gp120 binds to and activates one or more of MC1R, MC2R, MC3R and MC5R in vivo via the viral melanotropin epitope of SEQ ID NO:17. The identity of the melanocortin receptor stimulatory peptide(s) of HIV-1 gp120 is determined as described in Example 2 for MC5R.

Specifically, the present invention contemplates that HIV-1 envelope melanotropin epitope binding to the MC5R or other cell surface molecules on antigen-primed T cells, interferes with development of CD4+ CD25+ regulatory T cells. The reduction in regulatory T cell numbers manifests itself as an immunological over-stimulation. Indeed, evidence is accumulating that progression to AIDS is associated with a reduction of CD4+ CD25+ regulatory T cells, and an increase in T cell hyperactivation (Nixon et al., Microbes Infect, 7:1063-1065, 2005). On the other hand, elevated plasma levels of alpha-MSH (an endogenous ligand of MC5R as well as MC4R) are associated with increased survival in HIV infected patients. Specifically, circulating alpha-MSH concentration was greater in non-progressors than in progressors, while no such correlation was observed with circulating concentrations of interleukin-1-receptor antagonist and soluble tumor necrosis factor receptor (Airaghi et al., J Lab Clin Med, 133:309-315, 1999).

C. Central Nervous System Disease

HIV-related neuropsychiatric disorders are increasing in prevalence in the era of highly active antiretroviral therapy (HAART). It has been estimated that 30% of adults infected with HIV-1 are affected by minor cognitive motor disorder. In addition, despite HAART, 10% of infected patients will develop HIV-1 associated dementia. In fact in the United States, HIV-1 infection is the most common cause of dementia in young adults. Basic research has implicated the envelope glycoprotein of HIV-1, known as gp120, in HIV-related central nervous system (CNS) disease. Two lines of transgenic mice expressing HIV-1 gp120 and gp160 respectively in the CNS exhibit neuropathology (Toggas, Nature, 367:188-193, 1994; and Berrada et al., J Virol, 69:6770-6778, 1995). The present invention contemplates that the melanotropin epitope in HIV-1 gp120 or its degradation products binds to and activates one or more CNS cell surface molecules in vivo, thereby playing roles in the development of HIV-CNS disease. In particular, it is contemplated that chronic stimulation of CNS receptors by melanotropin epitope containing HIV-1 envelope fragments contributes to HIV-related neuropsychiatric disorders.

### IV. Generation of Viral Melanotropin Epitope-Reactive Antibodies

In some embodiments, antibodies are provided to allow for the detection of viral melanotropin epitope containing proteins. The antibodies may be prepared using various immunogens. In one embodiment, the immunogen is a synthetic peptide corresponding to the HIV-1_{SF2} melanotropin epitope (SEQ ID NO:17) used to generate antibodies that recognize HIV-1_{SF2} gp120. Such antibodies include, but are not limited to polyclonal, monoclonal, chimeric, single chain, Fab fragments, and Fab expression libraries.

In other preferred embodiments, the HIV-1 melanotropin epitope is defined by the generic octapeptide sequence WRXXXXXY (SEQ ID NO:31), where W at position 1, R at position 2 and Y at position 8 are invariant, and X can be any of the twenty natural amino acids humans and. Thus, the HIV-1 melanotropin epitopes of the present invention comprises at least 8 amino acids. However, in preferred embodiments the compositions and methods of the present invention comprise polypeptides consisting of the HIV-1_{SF2} melanotropin epitope (SEQ ID NO:17), polypeptides comprising the HIV-1_{SF2} melanotropin epitope (SEQ ID NO:17), and polypeptides consisting of or comprising variants thereof. In particularly preferred embodiments, the variants include HIV-1 melanotropin epitopes having from one to 22 (e.g., one, two, three, four, five, six, seven, eight, nine, 10, 11, 12, 13, 14, 15, 16, 17, 18, 19, 20, 21, or 22 conservative amino acid changes (e.g., substitutions) in relationship to SEQ ID NO:17, such that binding to one or both of MC4R or MC5R is maintained or substantially maintained. In further preferred embodiments, the variants include HIV-1 melanotropin epitopes have from one to 22 conservative or non-conservative amino acid changes (e.g., substitutions) in relationship to SEQ ID NO:17, such that binding to one or both of MC4R or MC5R is maintained or substantially maintained. In still further embodiments, the variants include HIV-1 melanotropin epitopes having from one to eight (e.g., one two, three, four, five, six, seven or eight) fewer N-terminal amino acids and/or from one to nine (e.g., one two, three, four, five, six, seven, eight or nine) fewer C-terminal amino acids in relationship to SEQ ID NO:17, such that binding to one or both of MC4R or MC5R is maintained or substantially maintained. In particularly preferred embodiments, the variants of the HIV-1 melanotropin epitopes include variants of the amino acid sequence set forth in SEQ ID NO:17, where W at position 9, R at position 10 and Y at position 10 are invariant, such that binding to one or both of MC4R or MC5R is maintained or substantially maintained.

Various procedures known in the art may be used for the production of polyclonal antibodies directed against the viral melanotropin epitope. For the production of antibody, various host animals can be immunized by injection with the peptide corresponding to the viral melanotropin epitope including but not limited to rabbits, mice, rats, sheep, goats, etc. In a preferred embodiment, the peptide is conjugated to an immunogenic carrier (e.g., diphtheria toxoid, bovine serum albumin, keyhole limpet hemocyanin, etc.). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, hemocyanins, dinitrophenol, and potentially useful human adjuvants such as BCG (Bacille Calmette-Guerin).

For preparation of monoclonal antibodies directed toward the viral melanotropin epitope, it is contemplated that any technique that provides for the production of antibody molecules by continuous cell lines in culture will find use with the present invention (See e.g., Harlow and Lane, Antibodies: A Laboratory Manual, Cold Spring Harbor Laboratory Press, Cold Spring Harbor, N.Y.). These include but are not limited to the hybridoma technique (Köhler and Milstein, Nature, 256:495-497, 1975; and Cote et al., Proc Natl Acad Sci, USA 80:2026-2030, 1983), as well as the trioma technique, and the EBV-hybridoma technique to produce human monoclonal antibodies (Cole et al, in Monoclonal Antibodies and Cancer Therapy, Alan R. Liss, Inc., pp. 77-96, 1985). In an additional embodiment of the invention, monoclonal antibodies are produced in germ-free animals utilizing technology such as that described in PCT/US90/02545).

Furthermore, it is contemplated that techniques described for the production of single chain antibodies (U.S. Pat. No. 4,946,778; herein incorporated by reference) will find use in producing viral melanotropin epitope-specific single chain antibodies. An additional embodiment of the invention utilizes the techniques described for the construction of Fab expression libraries (Huse et al., Science 246:1275-1281, 1989) to allow rapid and simple identification of monoclonal Fab fragments with the desired specificity for HIV-1 gp120.

It is contemplated that any technique suitable for producing antibody fragments will find use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody will be accomplished by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. In particular as is well known in the art, the immunogenic peptide should be provided free of the carrier molecule used in any immunization protocol. For example, if the peptide was conjugated to KLH, it may be conjugated to BSA, or used directly, in a screening assay.

The foregoing antibodies can be used in methods known in the art relating to the localization and structure of a viral melanotropin epitope (e.g., for Western blotting), measuring levels thereof in appropriate biological samples, etc. The antibodies can be used to detect a viral melanotropin epitope in a biological sample from an individual. The biological sample can be a biological fluid, such as, but not limited to, blood, serum, plasma, interstitial fluid, urine, cerebrospinal fluid, and the like, containing cells.

The biological samples can then be tested directly for the presence of a viral melanotropin epitope using an appropriate strategy (e.g., ELISA or radioimmunoassay) and format (e.g., microwells, dipstick, etc.). Alternatively, proteins in the sample can be size separated for instance by polyacrylamide gel electrophoresis (PAGE), in the presence or absence of sodium dodecyl sulfate (SDS), and the presence of a viral melanotropin epitope detected by immunoblotting (Western blotting). Immunoblotting techniques are generally more effective with antibodies generated against a peptide corresponding to an epitope of a protein, and hence, are particularly suited to the present invention.

### V. Drug Screening Using a Viral Melanotropin Epitope

The present invention provides methods and compositions for using a viral melanotropin epitope, HIV-1 gp120, or its degradation products as a target for screening drugs (e.g., small molecules, peptides, peptide mimetics, antibodies, etc.) that can inhibit aberrant stimulation of CNS cell surface molecules such as MC4R and/or MC5R by HIV-1 envelope fragments. As described below and elsewhere, several different assay systems (U.S. Pat. No. 5,908,609 to Lee et al., 1999; U.S. Pat. No. 6,100,048 to Cone et al., 2000; and U.S. Pat. No. 6,278,038 to Cone et al., 2001, all herein incorporated by reference in their entirety), can be designed and used to identify compounds or compositions that modulate MC4R and or MC5R activity. The systems described below may be formulated into kits. To this end, polypeptides comprising a viral melanotropin epitope can be packaged in a variety of containers (e.g., vials, tubes, microtitre well plates, bottles, and the like). Other reagents can be included in separate containers and provided with the kit (e.g., positive controls samples, negative control samples, melanocortin peptides including but not limited to alpha-MSH and ACTH derivatives), buffers, media, etc.

A. Cell-Based Assays

In accordance with the invention, a cell-based assay system can be used to screen for compounds that modulate viral melanotropin epitope stimulation of CNS cell surface molecules such as MC4R and or MC5R. In some embodiments, the compounds so identified are suitable for treating AIDS wasting syndrome and HIV-mediated T regulatory cell dysfunction. To this end, cells that endogenously express MC4R or MC5R can be used to screen for compounds. Alternatively, cell lines, such as 293 cells, COS cells, CHO cells, fibroblasts, and the like, genetically engineered to express the MC4R or MC5R can be used for screening purposes. Preferably, host cells genetically engineered to express a functional receptor that responds to activation by melanocortin peptides can be used as an endpoint in the assay (e.g., as measured by a chemical, physiological, biological, or phenotypic change, induction of a host cell gene or a reporter gene, change in cAMP levels, adenylyl cyclase activity, host cell G protein activity, extracellular acidification rate, host cell kinase activity, proliferation, differentiation, etc.).

To be useful in screening assays, the host cells expressing functional MC4R or MC5R should give a significant response to a MC4R or MC5R ligand, preferably greater than 5-fold induction over background. Host cells should preferably possess a number of characteristics, depending on the readout, to maximize the inductive response by melanocortin peptides, for example, for detecting a strong induction of a CRE reporter gene: (a) a low natural level of cAMP, (b) G proteins capable of interacting with the MC4R or MC5R, (c) a high level of adenylyl cyclase, (d) a high level of protein kinase A, (e) a low level of phosphodiesterases, and (f) a high level of cAMP response element binding protein would be advantageous. To increase response to melanocortin peptide, host cells could be engineered to express a greater amount of favorable factors or a lesser amount of unfavorable factors. In addition, alternative pathways for induction of the CRE reporter could be eliminated to reduce basal levels.

In utilizing such cell systems, the cells expressing the melanocortin receptor are exposed to a test compound or to vehicle controls (e.g., placebos). After exposure, the cells can be assayed to measure the expression and/or activity of components of the signal transduction pathway of the melanocortin receptor, or the activity of the signal transduction pathway itself can be assayed. For example, after exposure, cell lysates can be assayed for induction of cAMP. The ability of a test compound to increase levels of cAMP, above those levels seen with cells treated with a vehicle control, indicates that the test compound induces signal transduction mediated by the melanocortin receptor expressed by the host cell. In screening for compounds that may act as selective antagonists of melanocortin receptor stimulation by a viral melanotropin epitope, it is important to include ligands that activate the MCR (e.g., alpha-MSH, beta-MSH or ACTH) to test for inhibition of signal transduction by the test compound as compared to vehicle controls.

In a specific embodiment of the invention, constructs containing the cAMP responsive element linked to any of a variety of different reporter genes may be introduced into cells expressing the melanocortin receptor. Such reporter genes may include but is not limited to chloramphenicol acetyltransferase (CAT), luciferase, GUS, growth hormone, or placental alkaline phosphatase (SEAP). Following exposure of the cells to the test compound, the level of reporter gene expression may be quantitated to determine the test compound's ability to regulate receptor activity. Alkaline phosphatase assays are particularly usefull in the practice of the invention as the enzyme is secreted from the cell. Therefore, tissue culture supernatant may be assayed for secreted alkaline phosphatase. In addition, alkaline phosphatase activity may be measured by calorimetric, bioluminescent or chemilumenscent assays (Bronstein et al., Biotechniques, 17:172-177, 1994). Such assays provide a simple, sensitive easily automatable detection system for pharmaceutical screening.

When it is desired to discriminate between the melanocortin receptors and to identify compounds that selectively agonize or antagonize MC4R or MC5R, the assays described above should be conducted using a panel of host cells, each genetically engineered to express one of the melanocortin receptors (MC1R through MC5R). Expression of the human melanocortin receptors is preferred for drug discovery purposes. The cloning and characterization of each receptor has been described: MC1R and MC2R (Mountjoy et al., Science, 257:1248-1251, 1992; and Chhajlani and Wikberg, FEBS Lett, 309:417-420, 1992); MC3R (Roselli-Rehfuss et al., Proc Natl Acad Sci, USA, 90:8856-8860, 1993; and Gantz et al., J Biol Chem, 268:8246-8250, 1993); MC4R (Gantz et al., J Biol Chem, 268:15174-15179, 1993; and Mountjoy et al., Mol Endo, 8:1298-1308, 1994); and MC5R (Chhajlani et al., Biochem Biophys Res Commun, 195:866-873, 1993; and Gantz et al., Biochem Biophys Res Commun, 200:1214-1220, 1994), each of which is incorporated by reference herein in its entirety. Thus, each of the foregoing sequences can be utilized to engineer a cell or cell line that expresses one of the melanocortin receptors for use in screening assays described herein. To identify compounds that specifically or selectively regulate viral melanotropin epitope stimulation of MC4R activity, the activation or inhibition of MC4R is compared to the effect of the test compound on the other melanocortin receptors.

Alternatively, if the host cells express more than one melanocortin peptide receptor, the background signal produced by these receptors in response to melanocortin peptides must be “subtracted” from the signal (Gantz et al., supra). The background response produced by these non-MC4R melanocortin receptors can be determined by a number of methods, including elimination of MC4R activity by antisense, antibody or antagonist. In this regard, it should be noted that wild type CHO cells demonstrate a small endogenous response to melanocortin peptides, which must be subtracted from background. Alternatively, activity contributed from other melanocortin receptors could be eliminated by activating host cells with a MC4R-specific ligand, or including specific inhibitors of the other melanocortin receptors.

B. Non-Cell Based Assays

In addition to cell based assays, non-cell based assay systems may be used to identify compounds that inhibit viral melanotropin epitope binding to CNS cell surface molecules such as MC4R and/or MC5R.

For instance, isolated membranes may be used to identify compounds that prevent HIV-1 gp120 interaction with MC4R. In atypical experiment using isolated membranes, HEK293 cells may be genetically engineered to express the MC4R. Membranes can be harvested by standard techniques and used in an in vitro binding assay with a ¹²⁵I-labelled ligand (e.g., iodinated alpha-MSH, beta-MSH, gamma-MSH or ACTH). Specific binding is determined by comparison with binding assays performed in the presence of excess unlabelled ligand.

To identify compounds that inhibit viral melanotropin epitope binding to MC4R, membranes are incubated with labeled ligand in the presence or absence of a test compound. Compounds that bind to the receptor and compete with labeled ligand for binding to the membranes reduced the signal compared to the vehicle control samples.

Alternatively, soluble MC4R may be recombinantly expressed and utilized in non-cell based assays to identify compounds that inhibit viral melanotropin epitope binding to MC4R. Recombinantly expressed MC4R polypeptides or MC4R fusion proteins are prepared. In non-cell based assays the recombinantly expressed MC4R is attached to a solid substrate such as a test tube, microtitre well or a column, by means well known to those in the art. The test compounds are then assayed for their ability to prevent viral melanotropin epitope binding to the MC4R.

C. Candidate Compounds for Screening Purposes

The assays described above and elsewhere (U.S. Publication No. 20030105024 of Cone et al., 2003, herein incorporated by reference in its entirety) can identify compounds that affect viral melanotropin epitope-mediated MC4R activity. For example, compounds that affect viral melanotropin epitope-mediated MC4R activity include but are not limited to compounds that bind to the MC4R, inhibit binding of the pathogenic ligand, and either activate signal transduction (agonists) or block activation (antagonists), and compounds that bind to the pathogenic ligand of the MC4R and neutralize the pathogenic ligand (e.g., viral melanotropin epitope activity of HIV-1 gp120).

Some embodiments of the present invention provide mammalian melanocortin receptor agonists having the general formula: A-B-C-D-E-F-G-amide, wherein A is an aliphatic amino acid residue, including for example Leu, Ile, Nle and Met, as well as analogues and substituted derivatives thereof; B is an acidic amino acid residue, including for example Asp and Glu; C is a basic amino acid residue, such as His; D is an aromatic amino acid residue having a d-conformation, including d-Phe, d-Tyr and substituted derivatives thereof, E is a basic amino acid residue, for example Arg, Lys, homoArg, homoLys, and analogues or substituted derivatives thereof; F is Trp or substituted derivatives thereof; and G is Lys, homoLys or a substituted derivative thereof. In the peptide embodiments of the melanocortin receptor agonists of the invention, the peptide is cyclized by the formation of an amide bond between the side chain carboxyl group of the Asp or Glu residue at position B in the peptide, and the side chain amino group of the Lys or homoLys residue at position G.

Other embodiments of the present invention provide mammalian melanocortin receptor antagonists having the general formula: A-B-C-D-E-F-G-amide, wherein A is an aliphatic amino acid residue, including for example Leu, Ile, Nle and Met, as well as analogues and substituted derivatives thereof; B is an acidic amino acid residue, including for example Asp and Glu; C is a basic amino acid residue, such as His; D is an aromatic amino acid residue having a d-conformation, including d-Nal and substituted derivatives thereof; E is a basic amino acid residue, for example Arg, Lys, homoArg, homoLys, and analogues or substituted derivatives thereof; F is Trp or substituted derivatives thereof; and G is Lys, homoLys or a substituted derivative thereof. In the peptide embodiments of the melanocortin receptor antagonists of the invention, the peptide is cyclized by the formation of an amide bond between the side chain carboxyl group of the Asp or Glu residue at position B in the peptide, and the side chain amino group of the Lys or homoLys residue at position G. In preferred embodiments, the melanocortin receptor antagonists of the invention are agonists of the MC4 receptor and/or the MC5 receptor.

However, it should be noted that the assays suitable for use with the present invention also identify compounds that modulate MC4R or MC5R signal transduction (e.g., compounds that affect downstream signaling events, such as inhibitors or enhancers of G protein activities that participate in transducing the signal activated by ligand binding to the MC4R). The identification and use of such compounds that affect signaling events downstream of MC4R or MC5R are contemplated to modulate the effects of MC4R on the development AIDS wasting syndrome and the effects of MC5R on HIV-1 mediated regulatory T cell dysfunction.

The compounds that may be screened in accordance with the invention include, but are not limited to peptides, antibodies and fragments thereof, and other organic compounds (e.g., peptidomimetics) that preferably bind to HIV-1 gp120 to inhibit MC4R or MC5R stimulation by the viral melanotropin epitope contained therein. Compounds may include, but are not limited to, peptides such as, for example, soluble peptides, including but not limited to members of random peptide libraries (Lam et al., Nature 354: 82-84, 1991; and Houghten et al., 1991, Nature, 354: 84-86, 1991), and combinatorial chemistry-derived molecular library made of D- and/or L-configuration amino acids, phosphopeptides including, but not limited to, members of random or partially degenerate, directed phosphopeptide libraries (Songyang, Cell, 72: 767-778, 1993), antibodies including, but not limited to, polyclonal, monoclonal, humanized, anti-idiotypic, chimeric or single chain antibodies, and FAb, F(ab′)2 and FAb expression library fragments, and epitope-binding fragments thereof, and small organic or inorganic molecules. Other compounds that can be screened in accordance with the invention include but are not limited to small organic molecules that are able to, for example, cross the blood-brain barrier.

D. Additional CNS Cell Surface Molecules Bound By gp120 or its Degradation Products

The National Institute of Mental Health (NIMH) Psychoactive Drug Screening Program (PDSP) is utilized to identify additional CNS cell surface molecules bound by HIV-1 gp120 and its 50 kDa and 70 kDa thrombin-cleavage products, as well as synthetic peptides comprising a viral melanocortin eitope. In particular the present invention contemplates a receptorome screening strategy (Roth et al., Proc Natl Acad Sci USA, 99:11934-11939, 2002; and Armbruster and Roth, J Biol Chem, 280:5129-5132, 2005) to identify molecular targets for HIV-1 envelope. Both ligand binding screens and functional screens are employed to molecularly define HIV-1 envelope mediated psychoactivity. Specifically, various viral melanocortin epitope containing polypeptides are placed in contact with membrane preparations from cells expressing cloned receptors for determination of Ki values in a high throughput format. Cloned receptors include but are not limited to acetycholine receptors, adrenergic receptors, dopamine receptors, GABA receptors, histamine receptors, glutamate receptors, opiate receptors, peptide receptors, serotonin receptors, transporters, adenosine receptors, prostaglandin receptors, and imidazoline receptors (See, Table 3). The present invention contemplates that gp120 and one or both of its degradation products bind to and activate CNS receptors involved in body weight homeostasis, inflammation, and cognition.

### VI. Pharmaceutical Composition

The present invention further provides pharmaceutical compositions that comprise inhibitors or antagonists of a viral melanotropin epitope, including antibodies, alone or in combination with at least one other agent, such as a stabilizing compound, and may be administered in any sterile, biocompatible pharmaceutical carrier, including, but not limited to, saline, buffered saline, dextrose, and water.

The methods of the present invention find use in treating diseases or altering physiological states. Peptides can be administered to the patient intravenously in a pharmaceutically acceptable carrier such as physiological saline. Standard methods for intracellular delivery of peptides can be used (e.g., delivery via liposome). Such methods are well known to those of ordinary skill in the art. The formulations of this invention are useful for parenteral administration, such as intravenous, subcutaneous, intramuscular, and intraperitoneal. Therapeutic administration of a polypeptide intracellularly can also be accomplished using gene therapy as described above.

A. Dose Determinations

Toxicity and therapeutic efficacy of such compounds can be determined by standard pharmaceutical procedures in cell cultures or experimental animals, for example for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Normal dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; or 5,225,212, all of which are herein incorporated by reference). Those skilled in the art will employ different formulations for antibodies than for the small molecule inhibitors. Intracerebral administration may necessitate delivery in a manner different from intravenous injections.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

B. Formulations and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges of gelatin for use in an inhaler or insufflator may be formulated containing a powder mix of the compound and a suitable powder base such as lactose or starch. The compounds may be formulated for parenteral administration by injection (e.g., bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); U (units), mU (milliunits); min. (minutes); sec. (seconds); % (percent); kb (kilobase); bp (base pair); and PCR (polymerase chain reaction).

### EXAMPLE 1

### Identification of an HIV-1_{SF2} gp120 Peptide that Activates the Human Melanocortin-4 Receptor (hMC4R)

A series of peptides corresponding to the amino acid sequence of the fifth conserved domain of HIV-1_{SF2} gp120 were synthesized by Peninsula Labs (Belmont, Calif.).

Cell lines. The DNA coding sequence of the human melanocortin-4 receptor (hMC4R) were subcloned into pcDNAIneo vector (Invitrogen) and stably transfected into human embryonic kidney 293 (HEK293) cells using a modified calcium phosphate procedure (Chen and Okayama, Mol Cell Biol, 7:2745-2752, 1987). Stably transfected cells were selected in DMEM containing 10% newborn calf serum (NCS) and 1 mg/ml G418, and grown in medium supplemented with 500 μg/ml G418.

Adenylyl cyclase assays. Adenylyl cyclase activity was determined directly by measuring the ability of cells to convert [3H]adenine to [³H]cAMP following exposure of the cells to increasing doses of the gp120 peptides. Duplicate wells containing approximately 1×10⁶ cells were incubated for 2 h with 2.5 μCi of [³H]adenine in DMEM containing 10% NCS. After this the medium was aspirated and the cells solubilized with 1 ml of 2.5% perchloric acid, 0.1 mM cAMP. Lysates (0.8 ml) were removed, neutralized with 80 μl of 4.2 N KOH, and 0.42 ml of H₂O. The samples were mixed and the sediment was allowed to settle. A total of 0.1 ml of each lysate was counted in a beta-counter to determine the total amount of [³H]adenine incorporated into cells. cAMP was separated from the lysate following sequential chromatography over Dowex and alumina columns. cAMP was eluted from the alumina columns in 4 ml of Tris (pH 7.4) and counted in a beta-counter. Relative cyclase activity was calculated by determining the percentage of [³H]adenine converted into [³H]cAMP. The Kaleidagraph software package (Synergy Software, Reading, Pa.) was used for fitting curves to the data and calculating EC₅₀ and maximum response values.

Results. As shown in FIG. 8, the G25G peptide (#79) stimulated adenylyl cyclase activity in transgenic HEK-293 cells expressing the hMC4R. Specifically the HIV-1_{SF2} gp120 G25G peptide (GGGDMRDNWRSELYKYKVVKIEPLG, SEQ ID NO:17) stimulated cAMP production at a concentration of 10⁻⁶M, whereas several control peptides (e.g., #77 R12Q=RNSSSSGSSGAGQ, SEQ ID NO:23) did not stimulate hMC4R-induced adenylyl cyclase activity at the concentrations tested. In addition as shown in FIG. 9, the peptide #33 K13M corresponding to a portion of the G25G peptide synthesized in the reverse orientation also stimulated adenylyl cyclase activity in transgenic HEK-293 cells expressing the hMC4R. Specifically the K13M peptide (KYKYLESRWNDRM, SEQ ID NO:6) stimulated cAMP production at a concentration of 10⁻⁶M, whereas other gp120 peptides of similar lengths synthesized in either the forward (f) or reverse direction (r) did not stimulate hMC4R-induced adenylyl cyclase activity at the concentrations tested. Additional gp120 peptides tested:

#27fM13K = MRDNWRSELYKYK, SEQ ID NO: 24;
#29fR12K = RDNWRSELYKYK, SEQ ID NO: 25;
#31rK14D = KYKYLESRWNDRMD, SEQ ID NO: 26;
#35rK17G = KYKYLESRWNDRMDGGG, SEQ ID NO: 27;
#37fG17K = GGGDMRDNWRSELYKYK, SEQ ID NO: 28;
#39rK12R = KYKYLESRWNDR, SEQ ID NO: 29;
and
#41fD14K = DMRDNWRSELYKYK, SEQ ID NO: 30.

### EXAMPLE 2

### Identification of an HIV-1_{SF2} gp120 Peptide that Activates the Melanocortin-5 Receptor (MC5R)

Cell lines. The DNA coding sequence of the human melanocortin-5 receptor (hMC5R) is subcloned into a suitable eukaryotic expression vector such as pcDNAIneo vector (Invitrogen) and stably transfected into human embryonic kidney 293 (HEK293) cells using a modified calcium phosphate procedure (Chen and Okayama, Mol Cell Biol, 7:2745-2752, 1987) or other suitable technique. Stably transfected cells are selected in DMEM containing 10% newborn calf serum (NCS) and 1 mg/ml G418, and grown in medium supplemented with 500 μg/ml G418.

Adenylyl cyclase assays. Adenylyl cyclase activity is determined directly by measuring the ability of cells to convert [³H]adenine to [³H]cAMP following exposure of the cells to increasing doses of the gp120 peptides. Duplicate wells containing approximately 1×10⁶ cells are incubated for 2 h with 2.5 μCi of [³H]adenine in DMEM containing 10% NCS. After this the medium is aspirated and the cells solubilized with 1 ml of 2.5% perchloric acid, 0.1 mM cAMP. Lysates (0.8 ml) are removed, neutralized with 80 μl of 4.2 N KOH, and 0.42 ml of H₂O. The samples are mixed and the sediment is allowed to settle. A total of 0.1 ml of each lysate is counted in a beta-counter to determine the total amount of [³H]adenine incorporated into cells. cAMP is separated from the lysate following sequential chromatography over Dowex and alumina columns. cAMP is eluted from the alumina columns in 4 ml of Tris (pH 7.4) and counted in a beta-counter. Relative cyclase activity is calculated by determining the percentage of[³H]adenine converted into [³H]cAMP. The Kaleidagraph software package (Synergy Software, Reading, Pa.) is used for fitting curves to the data and calculating EC₅₀ and maximum response values.

### EXAMPLE 3

### Production and Detection of Viral Melanotropin Epitope-Reactive Antibodies

Peptide synthesis. A peptide corresponding to M13K (MRDNWRSELYKYK set forth as SEQ ID NO:24) was synthesized with carboxy terminal glycine and cysteine residues for coupling to a carrier. Similarly, peptides corresponding to G25G (SEQ ID NO:17) and K13M (SEQ ID NO:6) are synthesized after addition of a cysteine residue to the amino or carboxy terminus to facilitate coupling to a carrier such as keyhole limpet hemocyanin (KLH).

Generation of polyclonal antibodies. BALB/c mice are immunized intraperitoneally on days 0, 15, and 30 with approximately 50 μg of KLH-coupled peptide resuspended in 100 μl of phosphate-buffered saline (PBS) and emulsified with an equal volume of RIBI adjuvant (RIBI Immunochem Research Inc., Hamilton, Mont.). Blood is collected prior to initial immunization and after each boost from the tail vein, and the serum fraction is assayed for specific antibody content. HIV-1 Env-reactive antibody is measured by Western blot using strips derived from a sodium dodecyl sulfate-polyacrylamide gel electrophoresis (SDS-PAGE) gel of reduced peptides conjugated to a second carrier such as bovine serum albumin (BSA), recombinant soluble HIV-1 gp120, and/or lysates of an HIV-1 Env-expressing cell (e.g., infected cell). In an exemplary embodiment, BALB/c mice were immunized with M13K-KLH in Complete Freund's Adjuvant. After an eight-week rest period the mice were boosted with M13K-KLH in Incomplete Freund's Adjuvant, then re-boosted at two-week intervals with M13K-KLH in PBS. Serum from mice collected one week after each boost was tested against M13K-BSA in an ELISA. Antibody titers of 1:50 to 1:400 were obtained after the first boost, which increased to titers of 1:25,600 to 1:51,200 after subsequent boosts.

Monoclonal antibody (mAb) production. Splenocytes are harvested from an adult BALB/c mouse 3 days after boosting with the gp120 peptides conjugated to KLH. The splenocytes are fused to P3×63Ag8.653 myeloma cells at a 5:1 ratio, and hybridomas are selected in DMEM containing hypoxanthine-aminopterin-thymidine. Tissue culture supernatants are screened for HIV-1-reactivity as described for the generation of polyclonal antibodies.

Enzyme-linked immunosorbent assay (ELISA). HIV-1 envelope-specific antibodies induced by immunization can also be analysed by ELISA with recombinant soluble HIV-1 gp120 or gp120 peptide-BSA conjugate as the antigen. Immunlon-2 plates are coated with rgp120 (1 mg/ml) in carbonate buffer (15 mM Na₂CO_{3, 35} mM NaHCO₃ [pH 9.8]) overnight at 48° C. Solutions were aspirated, and the wells are filled with 100 ml of blocking buffer (Filter Paper Diluent; DuPont, Wilmington, Del.) containing 2.5% fetal bovine serum and incubated at 37° C. for 4 h. Plates are washed four times with PBS containing 0.05% Tween 20. Plasma samples are evaluated in duplicate at 1:50 dilution in wells containing borate buffer (0.1 M boric acid, 47 mM sodium borate, 75 mM NaCl, 0.05% [vol/vol] Tween 20) plus 2.5% fetal bovine serum. Color development is observed by using alkaline phosphatase-conjugated goat anti-monkey immunoglobulin G (Sigma Chemical Company, St. Louis, Mo.) followed by incubation with p-nitrophenylphosphate disodium hexahydrate (Sigma 104 phosphatase substrate) in diethanolamine buffer (0.9 M diethanolamine-7 mM MgCl2 [pH 9.8] with concentrated HCl). Absorbance is measured at 405 nm.

Neutralizing antibody detection. Antibodies that neutralize HIV-1 are measured in MT-2 cells by using a cell killing assay as described previously (Montefiori et al., J Clin Microbiol, 26:231-235, 1988). Virus stocks are prepared in H9 cells and titrated by p24 concentration and 50% tissue culture infective dose assay in MT-2 cells. Similar assays can be used to measure neutralizing antibodies against viral isolates in which the stock titer is determined and assays are performed in CEM×174 cells. Serum samples are heat inactivated for 1 h at 56° C. prior to assay.

### EXAMPLE 4

### HIV-1 gp120 Cleavage With Thrombin And Western Blot Analysis of Antibodies

HIV-1 gp120 of the SF162 isolate was obtained from the National Institutes of Health AIDS Reagent Program (catalog no. 7363). Thrombin from human plasma was obtained from Sigma (catalog no. T9010). Twenty microliters HIV-1 gp120 (1.2 μg/μl) and ten microliters of thrombin (30 Units/ml) were mixed and incubated at 37° C. for 2 hours. SDS sample buffer and reducing agent were added to the gp120-thrombin mixture, heated to 85° C. for 2 min and then analysed by SDS-PAGE (2 μg gp120/lane). After electrophoresis, the proteins were transferred to PVDF membrane for western blot analysis. The post-transfer gel was stained with Coomassie Blue and the post-transfer blot was stained with Ponceau S. The membrane was cut into strips, blocked overnight at 4° C. in a milk tween solution. Each strip was reacted with a control or a test antibody. The positive control was an HIV-1 gp120 monoclonal antibody, the negative control was cell culture medium and the test antibodies were cell culture supernatants of hybridoma clones obtained from an M13K-KLH immunized mouse. Bands of 120 kDa, 70 kDa and to a lesser extent 50 kDa were visualized in the Coomassie Blue stained gel. The 50 kDa band was obscured somewhat by the large band corresponding to the BSA stabilizer. The 120 kDa and 70 kDa bands were detected by the positive control antibody, whereas no bands were detected in the test antibody and negative control lanes at the dilutions tested.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in relevant fields, are intended to be within the scope of the following claims.

What is claimed is:

1. A method for detecting antibodies in a sample, comprising:
a) providing i) a sample, wherein said sample comprises antibodies; and ii) a polypeptide comprising an HIV-1 melanotropin epitope;
b) contacting said polypeptide with said sample under conditions suitable for binding said antibodies to said HIV-1 melanotropin epitope of said polypeptide; and
c) detecting HIV-1 melanotropin epitope-reactive antibodies bound to said polypeptide.

2. The method of claim 1, wherein said detecting comprises an enzyme-linked immunosorbent assay.

3. The method of claim 1, wherein said detecting comprises a Western blot.

4. The method of claim 1, wherein said sample is from an HIV-1 infected patient.

5. The method of claim 1, wherein said sample is from an AIDS vaccine recipient.

6. The method of claim 1, wherein said sample is from a hybridoma cell culture supernatant.

7. The method of claim 1, wherein said polypeptide comprises a 50 kDa fragment of HIV-1 gp120 produced by cleavage with thrombin.

8. The method of claim 1, wherein said polypeptide comprises a carrier.

9. The method of claim 1, wherein said polypeptide comprises the amino acid sequence set forth in SEQ ID NO:17 or SEQ ID NO:24.

10. The method of claim 1, wherein said polypeptide comprises an HIV-1 gp120 protein.

11. The method of claim 1, further comprising a step of detecting antibodies that neutralize HIV-1 in an in vitro assay.

12. A kit for detecting antibodies in a sample, comprising:
a) a polypeptide comprising an HIV-1 melanotropin epitope; and
b) instructions for detecting HIV-1 melanotropin epitope-reactive antibodies in a sample.

13. The kit of claim 12, further comprising a negative control polypeptide.

14. The kit of claim 13, further comprising a positive control antibody and a negative control antibody.

15. The kit of claim 12, wherein said polypeptide comprises the amino acid sequence set forth in SEQ ID NO:17 or SEQ ID NO:24.

16. A method for identifying a compound as an inhibitor of HIV-1 melanotropin epitope stimulation of a human melanocortin receptor (MCR), comprising;
a) providing: i) a cell line expressing a human MCR; ii) a polypeptide comprising an HIV-1 melanotropin epitope; and a iii) compound;
b) contacting said cell line with said polypeptide in the presence and absence of said compound under conditions suitable for stimulating said human MCR with said polypeptide, wherein stimulation of said human MCR in the absence but not the presence of said compound indicates that said compound is an inhibitor of said HIV-1 melanotropin epitope.

17. The method of claim 16, further identifying said compound as an inhibitor of human neuropeptide hormone stimulation of human MCR by step c) contacting said cell line with a human neuropeptide hormone in the presence and absence of said compound under conditions suitable for stimulating said MCR with said human neuropeptide hormone, wherein stimulation of said human MCR in the absence but not the presence of said compound indicates that said compound is an inhibitor of said human neuropeptide hormone.

18. The method of claim 16, wherein said human MCR is MC4R.

19. The method of claim 16, wherein said human MCR is selected from the group consisting of MC1R, MC2R, MC3R and MC5R.

20. The method of claim 18, wherein said cell line is a transgenic cell line expressing said MC4R.

21. The method of claim 16, wherein said polypeptide comprises the amino acid sequence set forth in SEQ ID NO:17 or SEQ ID NO:24.

22. The method of claim 17, wherein said human neuropeptide hormone is selected from the group consisting of alpha-melanocyte stimulating hormone (alpha-MSH), adrenocorticotrophin (ACTH), beta-melanocyte stimulating hormone (beta-MSH), and gamma-melanocyte stimulating hormone (gamma-MSH).
